(19) United States
(12) Patent Application Publication
(10) Pub. No.: US 2024/0033234 A1
(43) Pub. Date: Feb. 1, 2024

(54) THERAPEUTIC COMPOUNDS, FORMULATIONS, AND USE THEREOF

(71) Applicant: Tvardi Therapeutics, Inc., Sugar Land, TX (US)

(72) Inventors: Imran Alibhai, Sugar Land, TX (US); Sofia de Achaval, Missouri City, TX (US); Alan Millar, Holly Springs, NC (US)

(21) Appl. No.: 18/447,040

(22) Filed: Aug. 9, 2023

Related U.S. Application Data
(63) Continuation of application No. PCT/US2023/070440, filed on Jul. 18, 2023.
(60) Provisional application No. 63/369,055, filed on Jul. 21, 2022.

(51) Int. Cl. A61K 31/18

(52) U.S. Cl. CPC A61K 31/18

(57) ABSTRACT

The present disclosure, in part, is directed to compositions comprising a compound of formula (I):

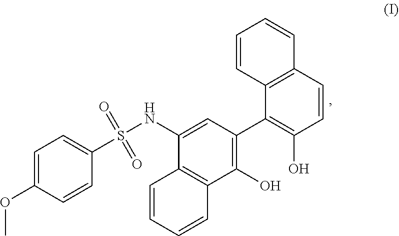 [SMILES: COc1ccc(S(=O)(=O)Nc2cc(-c3c(O)ccc4ccccc34)c(O)c3ccccc23)cc1]

wherein the compositions are substantially free of an impurity, processes of producing thereof, and uses thereof.

### CROSS REFERENCE

This application is a continuation of International Application No. PCT/US2023/070440, filed Jul. 18, 2023, which claims the benefit of and priority to U.S. Provisional Patent Application No. 63/369,055, filed Jul. 21, 2022, each of which are incorporated herein by reference in their entireties.

### SUMMARY

Disclosed herein, in certain embodiments, are compositions comprising a compound of formula (I), wherein the compositions are substantially free of an impurity, processes of producing said compositions, and uses thereof such as in treating, preventing, or reducing the risk or severity of certain diseases or disorders (e.g., cancer, fibrosis, and inflammatory diseases or disorders).

In certain embodiments, disclosed herein is a composition, comprising a compound of formula (I):

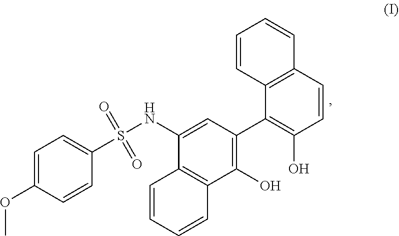 [SMILES: COc1ccc(S(=O)(=O)Nc2cc(-c3c(O)ccc4ccccc34)c(O)c3ccccc23)cc1]

wherein the composition is substantially free of an impurity.

In certain embodiments, disclosed herein is a composition, comprising a compound of formula (I), and a pharmaceutically acceptable carrier, wherein the composition is substantially free of an impurity.

In certain embodiments, disclosed herein is a composition, comprising a compound of formula (I), wherein the composition comprises no more than about 2% of a compound of formula (II).

In certain embodiments, disclosed herein is a composition, comprising a compound of formula (I), and a pharmaceutically acceptable carrier, wherein the composition comprises no more than about 2% of a compound of formula (II).

In certain embodiments, disclosed herein is a composition, comprising a compound of formula (I), wherein the composition comprises no more than about 0.05% of a compound of formula (III).

In certain embodiments, disclosed herein is a composition, comprising a compound of formula (I), and a pharmaceutically acceptable carrier, wherein the composition comprises no more than about 0.05% of a compound of formula (III).

In certain embodiments, provided herein is a method of treating a cancer in an individual in need thereof, the method comprising administering to the individual a therapeutically effective amount of the composition disclosed herein.

In certain embodiments, provided herein is a method of treating fibrosis in an individual in need thereof, the method comprising administering to the individual a therapeutically effective amount of the composition disclosed herein.

In certain embodiments, provided herein is a method of treating an inflammatory disease or disorder in an individual in need thereof, the method comprising administering to the individual a therapeutically effective amount of the composition disclosed herein.

In certain embodiments, provided herein is a method of treating chemotherapy-induced peripheral neuropathy in an individual in need thereof, the method comprising administering to the individual a therapeutically effective amount of the composition disclosed herein.

In certain embodiments, provided herein is a method of treating diabetic neuropathy in an individual in need thereof, the method comprising administering to the individual a therapeutically effective amount of the composition disclosed herein.

In certain embodiments, provided herein is a method of treating familial amyloid polyneuropathy in an individual in need thereof, the method comprising administering to the individual a therapeutically effective amount of the composition disclosed herein.

In certain embodiments, provided herein is a method of treating cachexia in an individual in need thereof, the method comprising administering to the individual a therapeutically effective amount of the composition disclosed herein.

In certain embodiments, provided herein is a method of treating anaphylaxis in an individual in need thereof, the method comprising administering to the individual a therapeutically effective amount of the composition disclosed herein.

In certain embodiments, provided herein is a method of treating non-alcoholic fatty liver disease or steatohepatitis in an individual in need thereof, the method comprising administering to the individual a therapeutically effective amount of the composition disclosed herein.

In certain embodiments, provided herein is a use of a composition disclosed herein for treating cancer in an individual in need thereof.

In certain embodiments, provided herein is a use of a composition disclosed herein for treating fibrosis in an individual in need thereof.

In certain embodiments, provided herein is a use of a composition disclosed herein for treating an inflammatory disease or disorder in an individual in need thereof.

In certain embodiments, provided herein is a use of a composition disclosed herein for treating chemotherapy-induced peripheral neuropathy in an individual in need thereof.

In certain embodiments, provided herein is a use of a composition disclosed herein for treating diabetic neuropathy in an individual in need thereof.

In certain embodiments, provided herein is a use of a composition disclosed herein for treating familial amyloid polyneuropathy in an individual in need thereof.

In certain embodiments, provided herein is a use of a composition disclosed herein for treating cachexia in an individual in need thereof.

In certain embodiments, provided herein is a use of a composition disclosed herein for treating anaphylaxis in an individual in need thereof.

In certain embodiments, provided herein is a use of a composition disclosed herein for treating anaphylaxis in an individual in need thereof.

In certain embodiments, provided herein is a use of a composition disclosed herein for treating non-alcoholic fatty liver disease or steatohepatitis in an individual in need thereof.

In certain embodiments, disclosed herein is a method of evaluating the purity of a composition comprising a compound of formula (I), comprising assaying the composition for the presence of a compound selected from the group consisting of a compound of formula (II), a compound of formula (III), and a compound of formula (IV).

In certain embodiments, disclosed herein is a process for preparing a compound of formula (I), substantially free of an impurity.

Other objects and advantages will become apparent to those skilled in the art from a consideration of the ensuing Detailed Description, Examples, and Claims.

### BRIEF DESCRIPTION OF THE DRAWINGS

FIG. 1 shows an exemplary ¹H-NMR spectrum of Compound 7 in DMSO-d6.

FIG. 2 shows an exemplary ¹³C-NMR spectrum of Compound 7 in DMSO-d6.

### DETAILED DESCRIPTION

In certain embodiments, the present disclosure provides compositions comprising a compound of formula (I),

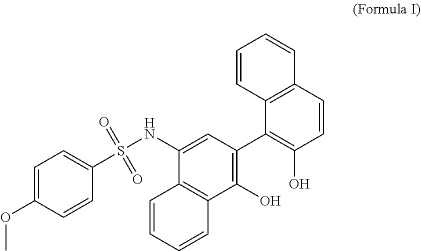 [SMILES: COc1ccc(S(=O)(=O)Nc2cc(-c3c(O)ccc4ccccc34)c(O)c3ccccc23)cc1]

wherein the compositions are substantially free of an impurity, processes of producing said compositions, as well as methods of treating, preventing, or reducing the risk or severity of certain diseases or disorders (e.g., cancer, fibrosis, inflammatory diseases or disorders) by administering said compositions.

### Definitions

As used herein the specification, “a” or “an” may mean one or more. As used herein, when used in conjunction with the word “comprising”, the words “a” or “an” may mean one or more than one. As used herein “another” may mean at least a second or more. Still further, the terms “having”, “including”, “containing” and “comprising” are interchangeable and one of skill in the art is cognizant that these terms are open ended terms. Some embodiments of the disclosure may consist of or consist essentially of one or more elements, method steps, and/or methods of the disclosure. It is contemplated that any method, compound, or composition described herein can be implemented with respect to any other method, compound, or composition described herein.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like.

As used herein, “pharmaceutically acceptable excipient” refers to any substance in a pharmaceutical formulation other than the active pharmaceutical ingredient(s). Exemplary pharmaceutical excipients include those that aid the manufacturing process; protect, support or enhance stability; increase bioavailability; or increase patient acceptability. They may also assist in product identification or enhance the overall safety or function of the product during storage or use. The terms “excipient” and “carrier” are used interchangeably herein.

As used herein, a “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In some embodiments, the subject is a human. In some embodiments, the subject is a non-human animal. The terms “human,” “patient,” “subject,” and “individual” are used interchangeably herein. None of these terms require the active supervision of medical personnel.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or reverses or slows the progression of the disease, disorder or condition (also “therapeutic treatment”).

In general, the “effective amount” of a compound refers to an amount sufficient to elicit the desired biological response. As will be appreciated by those of ordinary skill in this art, the effective amount of a compound of the disclosure may vary depending on such factors as the desired biological endpoint, the pharmacokinetics of the compound, the disease being treated, the mode of administration, and the age, weight, health, and condition of the subject. A “therapeutically effective amount” of a compound is an amount sufficient to provide a therapeutic benefit (e.g., treating, preventing, and/or ameliorating cancer in a subject, or inhibiting protein-protein interactions mediated by an SH2 domain in a subject, at a reasonable benefit/risk ratio applicable to any medical treatment) in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent. A “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent. A “prophylactic treatment” contemplates an action that occurs before a subject begins to suffer from the specified disease, disorder or condition.

The term “impurity” as used herein refers to a substance in the composition that is not the desired product (e.g., a compound of formula (I)) or an intended pharmaceutically acceptable carrier or excipient. Exemplary impurities include, but not limited to, synthetic impurities, intermediate impurities, isomers, oxidation products, hydrolyzed products, dimerization products and decomposition products of a compound of formula (I) and/or reactants or residual solvents used in the process of preparing a compound of formula (I). Exemplary structures of such impurities (compound of formula (II), compound of formula (III), compound of formula (IV)) are shown below:

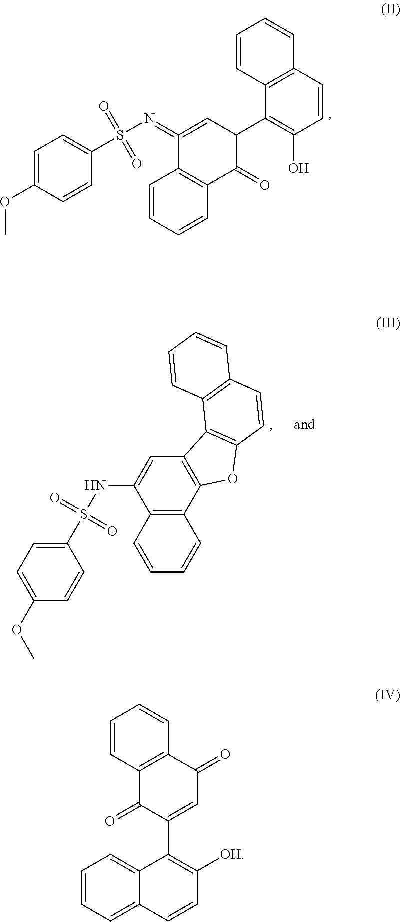

The term “substantially free” with respect to the compositions described herein relates to compositions comprising a compound of formula (I) that are at least a particular weight percent free of one or more impurities. Particular weight percentages of an impurity are 30%, 25%, 20%, 15%, 13%, 12%, 11%, 10%, 9%, 8%, 7%, 6%, 5%, 4%, 3%, 2%, 1%, 0.5%, 0.1%, 0.05, 0.01%, or 0.005% or any percentage between 0% and 1%, between 0% and 2%, between 0% and 5%, between 0% and 10%, or between 0% and 20%.

Ambient conditions of temperature as used herein refer to temperatures of between about 15 to about 30° C., or about 20 to about 30° C., for example, between about 20 to about 25° C.

### Compositions

Certain impurities may be incompatible with other substances in the composition (such as carriers) when formulated into pharmaceutical compositions comprising the active pharmaceutical ingredient and a pharmaceutically acceptable carrier; reduce shelf life of the composition; cause difficulties during formulation and use of the composition; cause physical and chemical instabilities of the compositions; lower therapeutic effects of the composition; show adverse biological effects; or change the odor, color, or taste of the composition. The compositions comprising a compound of formula (I) as described herein, in certain embodiments, are synthesized and processed in manners which produce compositions that are substantially free of impurities. In addition, the compositions comprising a compound of formula (I) as described herein, in certain embodiments, are synthesized and processed to produce sufficient amounts of the compound of formula (I)) (e.g., manufacturing scales, e.g., producing the compound of formula (I)) of greater than or equal to 1 kg per batch) while producing compositions that are substantially free of impurities.

In some embodiments, disclosed herein are compositions comprising a compound of formula (I):

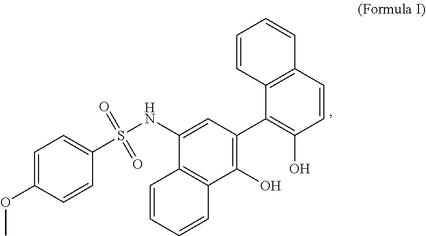 [SMILES: COc1ccc(S(=O)(=O)Nc2cc(-c3c(O)ccc4ccccc34)c(O)c3ccccc23)cc1]

or a pharmaceutically acceptable salt thereof, wherein the compositions are substantially free of an impurity.

In some embodiments, the impurity is a synthetic impurity, an intermediate impurity, an oxidation product, a hydrolyzed product of an impurity, a dimerization product, or a decomposition product of a compound of formula (I). In some embodiments, the impurity is a reactant or a residual solvent used in the process of preparing a compound of formula (I).

In some embodiments, the impurity is a compound of formula (II):

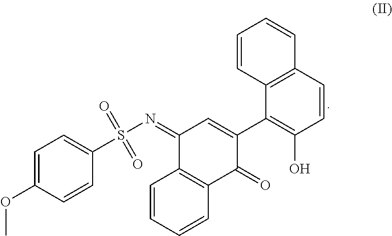 [SMILES: COc1ccc(S(=O)(=O)/N=C2/C=C(c3c(O)ccc4ccccc34)C(=O)c3ccccc32)cc1]

In some embodiments, the impurity is a compound of formula (III):

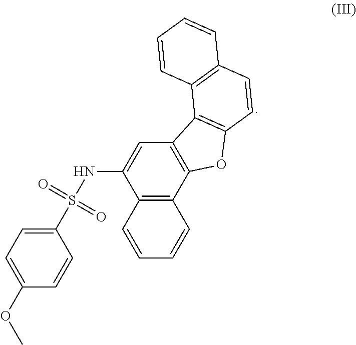 [SMILES: COc1ccc(S(=O)(=O)Nc2cc3c(oc4ccc5ccccc5c43)c3ccccc23)cc1]

In some embodiments, the impurity is a compound of formula (IV):

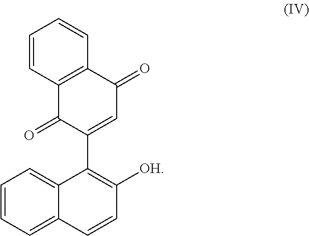 [SMILES: O=C1C=C(c2c(O)ccc3ccccc23)C(=O)c2ccccc21]

In some embodiments, the impurity is present in the composition at no more than about 2% area percentage by HPLC. In some embodiments, the impurity is present in the composition at no more than about 1% area percentage by HPLC. In some embodiments, the impurity is present in the composition at no more than about 0.5% area percentage by HPLC. In some embodiments, the impurity is present in the composition at no more than about 0.4% area percentage by HPLC. In some embodiments, the impurity is present in the composition at no more than about 0.3% area percentage by HPLC. In some embodiments, the impurity is present in the composition at no more than about 0.2% area percentage by HPLC. In some embodiments, the impurity is present in the composition at no more than about 0.1% area percentage by HPLC. In some embodiments, the impurity is present in the composition at no more than about 0.09% area percentage by HPLC. In some embodiments, the impurity is present in the composition at no more than about 0.08% area percentage by HPLC. In some embodiments, the impurity is present in the composition at no more than about 0.07% area percentage by HPLC. In some embodiments, the impurity is present in the composition at no more than about 0.06% area percentage by HPLC. In some embodiments, the impurity is present in the composition at no more than about 0.05% area percentage by HPLC. In some embodiments, the impurity is present in the composition at no more than about 0.04% area percentage by HPLC. In some embodiments, the impurity is present in the composition at no more than about 0.03% area percentage by HPLC. In some embodiments, the impurity is present in the composition at no more than about 0.02% area percentage by HPLC. In some embodiments, the impurity is present in the composition at no more than about 0.01% area percentage by HPLC. In some embodiments, the amount of impurity in the composition is undetectable by HPLC.

In some embodiments, the composition comprises no more than about 2% area percentage by HPLC of one or more impurities. In some embodiments, the composition comprises no more than about 1% area percentage by HPLC of one or more impurities. In some embodiments, the composition comprises no more than about 0.5% area percentage by HPLC of one or more impurities. In some embodiments, the composition comprises no more than about 0.4% area percentage by HPLC of one or more impurities. In some embodiments, the composition comprises no more than about 0.3% area percentage by HPLC of one or more impurities. In some embodiments, the composition comprises no more than about 0.2% area percentage by HPLC of one or more impurities. In some embodiments, the composition comprises no more than about 0.1% area percentage by HPLC of one or more impurities. In some embodiments, the composition comprises no more than about 0.09% area percentage by HPLC of one or more impurities. In some embodiments, the composition comprises no more than about 0.08% area percentage by HPLC of one or more impurities. In some embodiments, the composition comprises no more than about 0.07% area percentage by HPLC of one or more impurities. In some embodiments, the composition comprises no more than about 0.06% area percentage by HPLC of one or more impurities. In some embodiments, the composition comprises no more than about 0.05% area percentage by HPLC of one or more impurities. In some embodiments, the composition comprises no more than about 0.04% area percentage by HPLC of one or more impurities. In some embodiments, the composition comprises no more than about 0.03% area percentage by HPLC of one or more impurities. In some embodiments, the composition comprises no more than about 0.02% area percentage by HPLC of one or more impurities. In some embodiments, the composition comprises no more than about 0.01% area percentage by HPLC of one or more impurities.

In some embodiments, the composition comprising a compound of formula (I), or a pharmaceutically acceptable salt thereof, comprises no more than about 0.5% area percentage by HPLC of one or more impurities after storing the composition at about 40° C. and about 75% relative humidity for about 6 months. In some embodiments, the composition comprising a compound of formula (I), or a pharmaceutically acceptable salt thereof, comprises no more than about 0.4% area percentage by HPLC of one or more impurities after storing the composition at about 40° C. and about 75% relative humidity for about 6 months. In some embodiments, the composition comprising a compound of formula (I), or a pharmaceutically acceptable salt thereof, comprises no more than about 0.3% area percentage by HPLC of one or more impurities after storing the composition at about 40° C. and about 75% relative humidity for about 6 months. In some embodiments, the composition comprising a compound of formula (I), or a pharmaceutically acceptable salt thereof, comprises no more than about 0.2% area percentage by HPLC of one or more impurities after storing the composition at about 40° C. and about 75% relative humidity for about 6 months. In some embodiments, the composition comprising a compound of formula (I), or a pharmaceutically acceptable salt thereof, comprises no more than about 0.1% area percentage by HPLC of one or more impurities after storing the composition at about 40° C. and about 75% relative humidity for about 6 months.

In some embodiments, the composition comprising a compound of formula (I), or a pharmaceutically acceptable salt thereof, comprises no more than about 0.5% area percentage by HPLC of one or more impurities after storing the composition at about 40° C. and about 75% relative humidity for about 3 months. In some embodiments, the composition comprising a compound of formula (I), or a pharmaceutically acceptable salt thereof, comprises no more than about 0.4% area percentage by HPLC of one or more impurities after storing the composition at about 40° C. and about 75% relative humidity for about 3 months. In some embodiments, the composition comprising a compound of formula (I), or a pharmaceutically acceptable salt thereof, comprises no more than about 0.3% area percentage by HPLC of one or more impurities after storing the composition at about 40° C. and about 75% relative humidity for about 3 months. In some embodiments, the composition comprising a compound of formula (I), or a pharmaceutically acceptable salt thereof, comprises no more than about 0.2% area percentage by HPLC of one or more impurities after storing the composition at about 40° C. and about 75% relative humidity for about 3 months. In some embodiments, the composition comprising a compound of formula (I), or a pharmaceutically acceptable salt thereof, comprises no more than about 0.1% area percentage by HPLC of one or more impurities after storing the composition at about 40° C. and about 75% relative humidity for about 3 months.

In some embodiments, the composition comprising a compound of formula (I), or a pharmaceutically acceptable salt thereof, comprises no more than about 0.5% area percentage by HPLC of one or more impurities after storing the composition at about 40° C. and about 75% relative humidity for about 2 months. In some embodiments, the composition comprising a compound of formula (I), or a pharmaceutically acceptable salt thereof, comprises no more than about 0.4% area percentage by HPLC of one or more impurities after storing the composition at about 40° C. and about 75% relative humidity for about 2 months. In some embodiments, the composition comprising a compound of formula (I), or a pharmaceutically acceptable salt thereof, comprises no more than about 0.3% area percentage by HPLC of one or more impurities after storing the composition at about 40° C. and about 75% relative humidity for about 2 months. In some embodiments, the composition comprising a compound of formula (I), or a pharmaceutically acceptable salt thereof, comprises no more than about 0.2% area percentage by HPLC of one or more impurities after storing the composition at about 40° C. and about 75% relative humidity for about 2 months. In some embodiments, the composition comprising a compound of formula (I), or a pharmaceutically acceptable salt thereof, comprises no more than about 0.1% area percentage by HPLC of one or more impurities after storing the composition at about 40° C. and about 75% relative humidity for about 2 months.

In some embodiments, the composition comprising a compound of formula (I), or a pharmaceutically acceptable salt thereof, comprises no more than about 0.5% area percentage by HPLC of one or more impurities after storing the composition at about 40° C. and about 75% relative humidity for about 1 month. In some embodiments, the composition comprising a compound of formula (I), or a pharmaceutically acceptable salt thereof, comprises no more than about 0.4% area percentage by HPLC of one or more impurities after storing the composition at about 40° C. and about 75% relative humidity for about 1 month. In some embodiments, the composition comprising a compound of formula (I), or a pharmaceutically acceptable salt thereof, comprises no more than about 0.3% area percentage by HPLC of one or more impurities after storing the composition at about 40° C. and about 75% relative humidity for about 1 month. In some embodiments, the composition comprising a compound of formula (I), or a pharmaceutically acceptable salt thereof, comprises no more than about 0.2% area percentage by HPLC of one or more impurities after storing the composition at about 40° C. and about 75% relative humidity for about 1 month. In some embodiments, the composition comprising a compound of formula (I), or a pharmaceutically acceptable salt thereof, comprises no more than about 0.1% area percentage by HPLC of one or more impurities after storing the composition at about 40° C. and about 75% relative humidity for about 1 month.

In some embodiments, the composition comprising a compound of formula (I), or a pharmaceutically acceptable salt thereof, comprises no more than about 0.5% area percentage by HPLC of a compound of formula (II) after storing the composition at about 40° C. and about 75% relative humidity for about 3 months. In some embodiments, the composition comprising a compound of formula (I), or a pharmaceutically acceptable salt thereof, comprises no more than about 0.4% area percentage by HPLC of a compound of formula (II) after storing the composition at about 40° C. and about 75% relative humidity for about 3 months. In some embodiments, the composition comprising a compound of formula (I), or a pharmaceutically acceptable salt thereof, comprises no more than about 0.3% area percentage by HPLC of a compound of formula (II) after storing the composition at about 40° C. and about 75% relative humidity for about 3 months. In some embodiments, the composition comprising a compound of formula (I), or a pharmaceutically acceptable salt thereof, comprises no more than about 0.2% area percentage by HPLC of a compound of formula (II) after storing the composition at about 40° C. and about 75% relative humidity for about 3 months. In some embodiments, the composition comprising a compound of formula (I), or a pharmaceutically acceptable salt thereof, comprises no more than about 0.1% area percentage by HPLC of a compound of formula (II) after storing the composition at about and about 75% relative humidity for about 3 months.

In some embodiments, the composition comprising a compound of formula (I), or a pharmaceutically acceptable salt thereof, comprises no more than about 0.5% area percentage by HPLC of a compound of formula (II) after storing the composition at about 40° C. and about 75% relative humidity for about 2 months. In some embodiments, the composition comprising a compound of formula (I), or a pharmaceutically acceptable salt thereof, comprises no more than about 0.4% area percentage by HPLC of a compound of formula (II) after storing the composition at about 40° C. and about 75% relative humidity for about 2 months. In some embodiments, the composition comprising a compound of formula (I), or a pharmaceutically acceptable salt thereof, comprises no more than about 0.3% area percentage by HPLC of a compound of formula (II) after storing the composition at about 40° C. and about 75% relative humidity for about 2 months. In some embodiments, the composition comprising a compound of formula (I), or a pharmaceutically acceptable salt thereof, comprises no more than about 0.2% area percentage by HPLC of a compound of formula (II) after storing the composition at about 40° C. and about 75% relative humidity for about 2 months. In some embodiments, the composition comprising a compound of formula (I), or a pharmaceutically acceptable salt thereof, comprises no more than about 0.1% area percentage by HPLC of a compound of formula (II) after storing the composition at about 40° C. and about 75% relative humidity for about 2 months.

In some embodiments, the composition comprising a compound of formula (I), or a pharmaceutically acceptable salt thereof, comprises no more than about 0.5% area percentage by HPLC of a compound of formula (II) after storing the composition at about 40° C. and about 75% relative humidity for about 1 month. In some embodiments, the composition comprising a compound of formula (I), or a pharmaceutically acceptable salt thereof, comprises no more than about 0.4% area percentage by HPLC of a compound of formula (II) after storing the composition at about 40° C. and about 75% relative humidity for about 1 month. In some embodiments, the composition comprising a compound of formula (I), or a pharmaceutically acceptable salt thereof, comprises no more than about 0.3% area percentage by HPLC of a compound of formula (II) after storing the composition at about 40° C. and about 75% relative humidity for about 1 month. In some embodiments, the composition comprising a compound of formula (I), or a pharmaceutically acceptable salt thereof, comprises no more than about 0.2% area percentage by HPLC of a compound of formula (II) after storing the composition at about 40° C. and about 75% relative humidity for about 1 month. In some embodiments, the composition comprising a compound of formula (I), or a pharmaceutically acceptable salt thereof, comprises no more than about 0.1% area percentage by HPLC of a compound of formula (II) after storing the composition at about 40° C. and about 75% relative humidity for about 1 month.

In some embodiments, the composition comprising a compound of formula (I), or a pharmaceutically acceptable salt thereof, comprises no more than about 0.1% area percentage by HPLC of a compound of formula (III) after storing the composition at about 40° C. and about 75% relative humidity for about 3 months. In some embodiments, the composition comprising a compound of formula (I), or a pharmaceutically acceptable salt thereof, comprises no more than about 0.08% area percentage by HPLC of a compound of formula (III) after storing the composition at about 40° C. and about 75% relative humidity for about 3 months. In some embodiments, the composition comprising a compound of formula (I), or a pharmaceutically acceptable salt thereof, comprises no more than about 0.05% area percentage by HPLC of a compound of formula (III) after storing the composition at about 40° C. and about 75% relative humidity for about 3 months.

In some embodiments, the composition comprising a compound of formula (I), or a pharmaceutically acceptable salt thereof, comprises no more than about 0.1% area percentage by HPLC of a compound of formula (III) after storing the composition at about 40° C. and about 75% relative humidity for about 2 months. In some embodiments, the composition comprising a compound of formula (I), or a pharmaceutically acceptable salt thereof, comprises no more than about 0.08% area percentage by HPLC of a compound of formula (III) after storing the composition at about 40° C. and about 75% relative humidity for about 2 months. In some embodiments, the composition comprising a compound of formula (I), or a pharmaceutically acceptable salt thereof, comprises no more than about 0.05% area percentage by HPLC of a compound of formula (III) after storing the composition at about 40° C. and about 75% relative humidity for about 2 months.

In some embodiments, the composition comprising a compound of formula (I), or a pharmaceutically acceptable salt thereof, comprises no more than about 0.1% area percentage by HPLC of a compound of formula (III) after storing the composition at about 40° C. and about 75% relative humidity for about 1 month. In some embodiments, the composition comprising a compound of formula (I), or a pharmaceutically acceptable salt thereof, comprises no more than about 0.08% area percentage by HPLC of a compound of formula (III) after storing the composition at about 40° C. and about 75% relative humidity for about 1 month. In some embodiments, the composition comprising a compound of formula (I), or a pharmaceutically acceptable salt thereof, comprises no more than about 0.05% area percentage by HPLC of a compound of formula (III) after storing the composition at about 40° C. and about 75% relative humidity for about 1 month.

In some embodiments, the composition comprising a compound of formula (I), or a pharmaceutically acceptable salt thereof, comprises no more than about 0.5% area percentage by HPLC of a compound of formula (IV) after storing the composition at about 40° C. and about 75% relative humidity for about 3 months. In some embodiments, the composition comprising a compound of formula (I), or a pharmaceutically acceptable salt thereof, comprises no more than about 0.4% area percentage by HPLC of a compound of formula (IV) after storing the composition at about 40° C. and about 75% relative humidity for about 3 months. In some embodiments, the composition comprising a compound of formula (I), or a pharmaceutically acceptable salt thereof, comprises no more than about 0.3% area percentage by HPLC of a compound of formula (IV) after storing the composition at about 40° C. and about 75% relative humidity for about 3 months. In some embodiments, the composition comprising a compound of formula (I), or a pharmaceutically acceptable salt thereof, comprises no more than about 0.2% area percentage by HPLC of a compound of formula (IV) after storing the composition at about 40° C. and about 75% relative humidity for about 3 months. In some embodiments, the composition comprising a compound of formula (I), or a pharmaceutically acceptable salt thereof, comprises no more than about 0.1% area percentage by HPLC of a compound of formula (IV) after storing the composition at about 40° C. and about 75% relative humidity for about 3 months.

In some embodiments, the composition comprising a compound of formula (I), or a pharmaceutically acceptable salt thereof, comprises no more than about 0.5% area percentage by HPLC of a compound of formula (IV) after storing the composition at about 40° C. and about 75% relative humidity for about 2 months. In some embodiments, the composition comprising a compound of formula (I), or a pharmaceutically acceptable salt thereof, comprises no more than about 0.4% area percentage by HPLC of a compound of formula (IV) after storing the composition at about 40° C. and about 75% relative humidity for about 2 months. In some embodiments, the composition comprising a compound of formula (I), or a pharmaceutically acceptable salt thereof, comprises no more than about 0.3% area percentage by HPLC of a compound of formula (IV) after storing the composition at about 40° C. and about 75% relative humidity for about 2 months. In some embodiments, the composition comprising a compound of formula (I), or a pharmaceutically acceptable salt thereof, comprises no more than about 0.2% area percentage by HPLC of a compound of formula (IV) after storing the composition at about 40° C. and about 75% relative humidity for about 2 months. In some embodiments, the composition comprising a compound of formula (I), or a pharmaceutically acceptable salt thereof, comprises no more than about 0.1% area percentage by HPLC of a compound of formula (IV) after storing the composition at about 40° C. and about 75% relative humidity for about 2 months.

In some embodiments, the composition comprising a compound of formula (I), or a pharmaceutically acceptable salt thereof, comprises no more than about 0.5% area percentage by HPLC of a compound of formula (IV) after storing the composition at about 40° C. and about 75% relative humidity for about 1 month. In some embodiments, the composition comprising a compound of formula (I), or a pharmaceutically acceptable salt thereof, comprises no more than about 0.4% area percentage by HPLC of a compound of formula (IV) after storing the composition at about 40° C. and about 75% relative humidity for about 1 month. In some embodiments, the composition comprising a compound of formula (I), or a pharmaceutically acceptable salt thereof, comprises no more than about 0.3% area percentage by HPLC of a compound of formula (IV) after storing the composition at about 40° C. and about 75% relative humidity for about 1 month. In some embodiments, the composition comprising a compound of formula (I), or a pharmaceutically acceptable salt thereof, comprises no more than about 0.2% area percentage by HPLC of a compound of formula (IV) after storing the composition at about 40° C. and about 75% relative humidity for about 1 month. In some embodiments, the composition comprising a compound of formula (I), or a pharmaceutically acceptable salt thereof, comprises no more than about 0.1% area percentage by HPLC of a compound of formula (IV) after storing the composition at about 40° C. and about 75% relative humidity for about 1 month.

In some embodiments, the composition comprising a compound of formula (I), or a pharmaceutically acceptable salt thereof, comprises no more than about 0.3% area percentage by HPLC of one or more impurities after storing the composition at about 25° C. and about 60% relative humidity for about 24 months. In some embodiments, the composition comprising a compound of formula (I), or a pharmaceutically acceptable salt thereof, comprises no more than about 0.2% area percentage by HPLC of one or more impurities after storing the composition at about 25° C. and about 60% relative humidity for about 24 months. In some embodiments, the composition comprising a compound of formula (I), or a pharmaceutically acceptable salt thereof, comprises no more than about 0.1% area percentage by HPLC of one or more impurities after storing the composition at about 25° C. and about 60% relative humidity for about 24 months.

In some embodiments, the composition comprising a compound of formula (I), or a pharmaceutically acceptable salt thereof, comprises no more than about 0.3% area percentage by HPLC of one or more impurities after storing the composition at about 25° C. and about 60% relative humidity for about 12 months. In some embodiments, the composition comprising a compound of formula (I), or a pharmaceutically acceptable salt thereof, comprises no more than about 0.2% area percentage by HPLC of one or more impurities after storing the composition at about 25° C. and about 60% relative humidity for about 12 months. In some embodiments, the composition comprising a compound of formula (I), or a pharmaceutically acceptable salt thereof, comprises no more than about 0.1% area percentage by HPLC of one or more impurities after storing the composition at about 25° C. and about 60% relative humidity for about 12 months.

In some embodiments, the composition comprising a compound of formula (I), or a pharmaceutically acceptable salt thereof, comprises no more than about 0.3% area percentage by HPLC of one or more impurities after storing the composition at about 25° C. and about 60% relative humidity for about 6 months. In some embodiments, the composition comprising a compound of formula (I), or a pharmaceutically acceptable salt thereof, comprises no more than about 0.2% area percentage by HPLC of one or more impurities after storing the composition at about 40° C. and about 75% relative humidity for about 6 months. In some embodiments, the composition comprising a compound of formula (I), or a pharmaceutically acceptable salt thereof, comprises no more than about 0.1% area percentage by HPLC of one or more impurities after storing the composition at about 25° C. and about 60% relative humidity for about 6 months.

In some embodiments, the composition comprising a compound of formula (I), or a pharmaceutically acceptable salt thereof, comprises no more than about 0.3% area percentage by HPLC of one or more impurities after storing the composition at about 25° C. and about 60% relative humidity for about 3 months. In some embodiments, the composition comprising a compound of formula (I), or a pharmaceutically acceptable salt thereof, comprises no more than about 0.2% area percentage by HPLC of one or more impurities after storing the composition at about 40° C. and about 75% relative humidity for about 3 months. In some embodiments, the composition comprising a compound of formula (I), or a pharmaceutically acceptable salt thereof, comprises no more than about 0.1% area percentage by HPLC of one or more impurities after storing the composition at about 25° C. and about 60% relative humidity for about 3 months.

In some embodiments, the composition comprising a compound of formula (I), or a pharmaceutically acceptable salt thereof, comprises no more than about 0.3% area percentage by HPLC of one or more impurities after storing the composition at about 25° C. and about 60% relative humidity for about 2 months. In some embodiments, the composition comprising a compound of formula (I), or a pharmaceutically acceptable salt thereof, comprises no more than about 0.2% area percentage by HPLC of one or more impurities after storing the composition at about 25° C. and about 60% relative humidity for about 2 months. In some embodiments, the composition comprising a compound of formula (I), or a pharmaceutically acceptable salt thereof, comprises no more than about 0.1% area percentage by HPLC of one or more impurities after storing the composition at about 25° C. and about 60% relative humidity for about 2 months.

In some embodiments, the composition comprising a compound of formula (I), or a pharmaceutically acceptable salt thereof, comprises no more than about 0.3% area percentage by HPLC of one or more impurities after storing the composition at about 25° C. and about 60% relative humidity for about 1 month. In some embodiments, the composition comprising a compound of formula (I), or a pharmaceutically acceptable salt thereof, comprises no more than about 0.2% area percentage by HPLC of one or more impurities after storing the composition at about 25° C. and about 60% relative humidity for about 1 month. In some embodiments, the composition comprising a compound of formula (I), or a pharmaceutically acceptable salt thereof, comprises no more than about 0.1% area percentage by HPLC of one or more impurities after storing the composition at about 25° C. and about 60% relative humidity for about 1 month.

In some embodiments, the composition comprising a compound of formula (I), or a pharmaceutically acceptable salt thereof, comprises no more than about 0.3% area percentage by HPLC of a compound of formula (II) after storing the composition at about 25° C. and about 60% relative humidity for about 24 months. In some embodiments, the composition comprising a compound of formula (I), or a pharmaceutically acceptable salt thereof, comprises no more than about 0.2% area percentage by HPLC of a compound of formula (II) after storing the composition at about 25° C. and about 60% relative humidity for about 24 months. In some embodiments, the composition comprising a compound of formula (I), or a pharmaceutically acceptable salt thereof, comprises no more than about 0.1% area percentage by HPLC of a compound of formula (II) after storing the composition at about 25° C. and about 60% relative humidity for about 24 months.

In some embodiments, the composition comprising a compound of formula (I), or a pharmaceutically acceptable salt thereof, comprises no more than about 0.3% area percentage by HPLC of a compound of formula (II) after storing the composition at about 25° C. and about 60% relative humidity for about 12 months. In some embodiments, the composition comprising a compound of formula (I), or a pharmaceutically acceptable salt thereof, comprises no more than about 0.2% area percentage by HPLC of a compound of formula (II) after storing the composition at about 25° C. and about 60% relative humidity for about 12 months. In some embodiments, the composition comprising a compound of formula (I), or a pharmaceutically acceptable salt thereof, comprises no more than about 0.1% area percentage by HPLC of a compound of formula (II) after storing the composition at about 25° C. and about 60% relative humidity for about 12 months.

In some embodiments, the composition comprising a compound of formula (I), or a pharmaceutically acceptable salt thereof, comprises no more than about 0.3% area percentage by HPLC of a compound of formula (II) after storing the composition at about 25° C. and about 60% relative humidity for about 6 months. In some embodiments, the composition comprising a compound of formula (I), or a pharmaceutically acceptable salt thereof, comprises no more than about 0.2% area percentage by HPLC of a compound of formula (II) after storing the composition at about 25° C. and about 60% relative humidity for about 6 months. In some embodiments, the composition comprising a compound of formula (I), or a pharmaceutically acceptable salt thereof, comprises no more than about 0.1% area percentage by HPLC of a compound of formula (II) after storing the composition at about 25° C. and about 60% relative humidity for about 6 months.

In some embodiments, the composition comprising a compound of formula (I), or a pharmaceutically acceptable salt thereof, comprises no more than about 0.3% area percentage by HPLC of a compound of formula (II) after storing the composition at about 25° C. and about 60% relative humidity for about 3 months. In some embodiments, the composition comprising a compound of formula (I), or a pharmaceutically acceptable salt thereof, comprises no more than about 0.2% area percentage by HPLC of a compound of formula (II) after storing the composition at about 25° C. and about 60% relative humidity for about 3 months. In some embodiments, the composition comprising a compound of formula (I), or a pharmaceutically acceptable salt thereof, comprises no more than about 0.1% area percentage by HPLC of a compound of formula (II) after storing the composition at about 25° C. and about 60% relative humidity for about 3 months.

In some embodiments, the composition comprising a compound of formula (I), or a pharmaceutically acceptable salt thereof, comprises no more than about 0.3% area percentage by HPLC of a compound of formula (II) after storing the composition at about 25° C. and about 60% relative humidity for about 2 months. In some embodiments, the composition comprising a compound of formula (I), or a pharmaceutically acceptable salt thereof, comprises no more than about 0.2% area percentage by HPLC of a compound of formula (II) after storing the composition at about 25° C. and about 60% relative humidity for about 2 months. In some embodiments, the composition comprising a compound of formula (I), or a pharmaceutically acceptable salt thereof, comprises no more than about 0.1% area percentage by HPLC of a compound of formula (II) after storing the composition at about 25° C. and about 60% relative humidity for about 2 months.

In some embodiments, the composition comprising a compound of formula (I), or a pharmaceutically acceptable salt thereof, comprises no more than about 0.3% area percentage by HPLC of a compound of formula (II) after storing the composition at about 25° C. and about 60% relative humidity for about 1 month. In some embodiments, the composition comprising a compound of formula (I), or a pharmaceutically acceptable salt thereof, comprises no more than about 0.2% area percentage by HPLC of a compound of formula (II) after storing the composition at about 25° C. and about 60% relative humidity for about 1 month. In some embodiments, the composition comprising a compound of formula (I), or a pharmaceutically acceptable salt thereof, comprises no more than about 0.1% area percentage by HPLC of a compound of formula (II) after storing the composition at about 25° C. and about 60% relative humidity for about 1 month.

In some embodiments, the composition comprising a compound of formula (I), or a pharmaceutically acceptable salt thereof, comprises no more than about 0.1% area percentage by HPLC of a compound of formula (III) after storing the composition at about 25° C. and about 60% relative humidity for about 24 months. In some embodiments, the composition comprising a compound of formula (I), or a pharmaceutically acceptable salt thereof, comprises no more than about 0.08% area percentage by HPLC of a compound of formula (III) after storing the composition at about 25° C. and about 60% relative humidity for about 24 months. In some embodiments, the composition comprising a compound of formula (I), or a pharmaceutically acceptable salt thereof, comprises no more than about 0.05% area percentage by HPLC of a compound of formula (III) after storing the composition at about 25° C. and about 60% relative humidity for about 24 months.

In some embodiments, the composition comprising a compound of formula (I), or a pharmaceutically acceptable salt thereof, comprises no more than about 0.1% area percentage by HPLC of a compound of formula (III) after storing the composition at about 25° C. and about 60% relative humidity for about 12 months. In some embodiments, the composition comprising a compound of formula (I), or a pharmaceutically acceptable salt thereof, comprises no more than about 0.08% area percentage by HPLC of a compound of formula (III) after storing the composition at about 25° C. and about 60% relative humidity for about 12 months. In some embodiments, the composition comprising a compound of formula (I), or a pharmaceutically acceptable salt thereof, comprises no more than about 0.05% area percentage by HPLC of a compound of formula (III) after storing the composition at about 25° C. and about 60% relative humidity for about 12 months.

In some embodiments, the composition comprising a compound of formula (I), or a pharmaceutically acceptable salt thereof, comprises no more than about 0.1% area percentage by HPLC of a compound of formula (III) after storing the composition at about 25° C. and about 60% relative humidity for about 6 months. In some embodiments, the composition comprising a compound of formula (I), or a pharmaceutically acceptable salt thereof, comprises no more than about 0.08% area percentage by HPLC of a compound of formula (III) after storing the composition at about 25° C. and about 60% relative humidity for about 6 months. In some embodiments, the composition comprising a compound of formula (I), or a pharmaceutically acceptable salt thereof, comprises no more than about 0.05% area percentage by HPLC of a compound of formula (III) after storing the composition at about 25° C. and about 60% relative humidity for about 6 months.

In some embodiments, the composition comprising a compound of formula (I), or a pharmaceutically acceptable salt thereof, comprises no more than about 0.1% area percentage by HPLC of a compound of formula (III) after storing the composition at about 25° C. and about 60% relative humidity for about 3 months. In some embodiments, the composition comprising a compound of formula (I), or a pharmaceutically acceptable salt thereof, comprises no more than about 0.08% area percentage by HPLC of a compound of formula (III) after storing the composition at about 25° C. and about 60% relative humidity for about 3 months. In some embodiments, the composition comprising a compound of formula (I), or a pharmaceutically acceptable salt thereof, comprises no more than about 0.05% area percentage by HPLC of a compound of formula (III) after storing the composition at about 25° C. and about 60% relative humidity for about 3 months.

In some embodiments, the composition comprising a compound of formula (I), or a pharmaceutically acceptable salt thereof, comprises no more than about 0.1% area percentage by HPLC of a compound of formula (III) after storing the composition at about 25° C. and about 60% relative humidity for about 2 months. In some embodiments, the composition comprising a compound of formula (I), or a pharmaceutically acceptable salt thereof, comprises no more than about 0.08% area percentage by HPLC of a compound of formula (III) after storing the composition at about 25° C. and about 60% relative humidity for about 2 months. In some embodiments, the composition comprising a compound of formula (I), or a pharmaceutically acceptable salt thereof, comprises no more than about 0.05% area percentage by HPLC of a compound of formula (III) after storing the composition at about 25° C. and about 60% relative humidity for about 2 months.

In some embodiments, the composition comprising a compound of formula (I), or a pharmaceutically acceptable salt thereof, comprises no more than about 0.1% area percentage by HPLC of a compound of formula (III) after storing the composition at about 25° C. and about 60% relative humidity for about 1 month. In some embodiments, the composition comprising a compound of formula (I), or a pharmaceutically acceptable salt thereof, comprises no more than about 0.08% area percentage by HPLC of a compound of formula (III) after storing the composition at about 25° C. and about 60% relative humidity for about 1 month. In some embodiments, the composition comprising a compound of formula (I), or a pharmaceutically acceptable salt thereof, comprises no more than about 0.05% area percentage by HPLC of a compound of formula (III) after storing the composition at about 25° C. and about 60% relative humidity for about 1 month.

In some embodiments, the composition comprising a compound of formula (I), or a pharmaceutically acceptable salt thereof, comprises no more than about 0.5% area percentage by HPLC of a compound of formula (IV) after storing the composition at about 25° C. and about 60% relative humidity for about 24 months. In some embodiments, the composition comprising a compound of formula (I), or a pharmaceutically acceptable salt thereof, comprises no more than about 0.4% area percentage by HPLC of a compound of formula (IV) after storing the composition at about 25° C. and about 60% relative humidity for about 24 months. In some embodiments, the composition comprising a compound of formula (I), or a pharmaceutically acceptable salt thereof, comprises no more than about 0.3% area percentage by HPLC of a compound of formula (IV) after storing the composition at about 25° C. and about 60% relative humidity for about 24 months. In some embodiments, the composition comprising a compound of formula (I), or a pharmaceutically acceptable salt thereof, comprises no more than about 0.2% area percentage by HPLC of a compound of formula (IV) after storing the composition at about 25° C. and about 60% relative humidity for about 24 months. In some embodiments, the composition comprising a compound of formula (I), or a pharmaceutically acceptable salt thereof, comprises no more than about 0.1% area percentage by HPLC of a compound of formula (IV) after storing the composition at about 25° C. and about 60% relative humidity for about 24 months.

In some embodiments, the composition comprising a compound of formula (I), or a pharmaceutically acceptable salt thereof, comprises no more than about 0.5% area percentage by HPLC of a compound of formula (IV) after storing the composition at about 25° C. and about 60% relative humidity for about 12 months. In some embodiments, the composition comprising a compound of formula (I), or a pharmaceutically acceptable salt thereof, comprises no more than about 0.4% area percentage by HPLC of a compound of formula (IV) after storing the composition at about 25° C. and about 60% relative humidity for about 12 months. In some embodiments, the composition comprising a compound of formula (I), or a pharmaceutically acceptable salt thereof, comprises no more than about 0.3% area percentage by HPLC of a compound of formula (IV) after storing the composition at about 25° C. and about 60% relative humidity for about 12 months. In some embodiments, the composition comprising a compound of formula (I), or a pharmaceutically acceptable salt thereof, comprises no more than about 0.2% area percentage by HPLC of a compound of formula (IV) after storing the composition at about 25° C. and about 60% relative humidity for about 12 months. In some embodiments, the composition comprising a compound of formula (I), or a pharmaceutically acceptable salt thereof, comprises no more than about 0.1% area percentage by HPLC of a compound of formula (IV) after storing the composition at about 25° C. and about 60% relative humidity for about 12 months.

In some embodiments, the composition comprising a compound of formula (I), or a pharmaceutically acceptable salt thereof, comprises no more than about 0.5% area percentage by HPLC of a compound of formula (IV) after storing the composition at about 25° C. and about 60% relative humidity for about 6 months. In some embodiments, the composition comprising a compound of formula (I), or a pharmaceutically acceptable salt thereof, comprises no more than about 0.4% area percentage by HPLC of a compound of formula (IV) after storing the composition at about 25° C. and about 60% relative humidity for about 6 months. In some embodiments, the composition comprising a compound of formula (I), or a pharmaceutically acceptable salt thereof, comprises no more than about 0.3% area percentage by HPLC of a compound of formula (IV) after storing the composition at about 25° C. and about 60% relative humidity for about 6 months. In some embodiments, the composition comprising a compound of formula (I), or a pharmaceutically acceptable salt thereof, comprises no more than about 0.2% area percentage by HPLC of a compound of formula (IV) after storing the composition at about 25° C. and about 60% relative humidity for about 6 months. In some embodiments, the composition comprising a compound of formula (I), or a pharmaceutically acceptable salt thereof, comprises no more than about 0.1% area percentage by HPLC of a compound of formula (IV) after storing the composition at about 25° C. and about 60% relative humidity for about 6 months.

In some embodiments, the composition comprising a compound of formula (I), or a pharmaceutically acceptable salt thereof, comprises no more than about 0.5% area percentage by HPLC of a compound of formula (IV) after storing the composition at about 25° C. and about 60% relative humidity for about 3 months. In some embodiments, the composition comprising a compound of formula (I), or a pharmaceutically acceptable salt thereof, comprises no more than about 0.4% area percentage by HPLC of a compound of formula (IV) after storing the composition at about 25° C. and about 60% relative humidity for about 3 months. In some embodiments, the composition comprising a compound of formula (I), or a pharmaceutically acceptable salt thereof, comprises no more than about 0.3% area percentage by HPLC of a compound of formula (IV) after storing the composition at about 25° C. and about 60% relative humidity for about 3 months. In some embodiments, the composition comprising a compound of formula (I), or a pharmaceutically acceptable salt thereof, comprises no more than about 0.2% area percentage by HPLC of a compound of formula (IV) after storing the composition at about 25° C. and about 60% relative humidity for about 3 months. In some embodiments, the composition comprising a compound of formula (I), or a pharmaceutically acceptable salt thereof, comprises no more than about 0.1% area percentage by HPLC of a compound of formula (IV) after storing the composition at about 25° C. and about 60% relative humidity for about 3 months.

In some embodiments, the composition comprising a compound of formula (I), or a pharmaceutically acceptable salt thereof, comprises no more than about 0.5% area percentage by HPLC of a compound of formula (IV) after storing the composition at about 25° C. and about 60% relative humidity for about 2 months. In some embodiments, the composition comprising a compound of formula (I), or a pharmaceutically acceptable salt thereof, comprises no more than about 0.4% area percentage by HPLC of a compound of formula (IV) after storing the composition at about 25° C. and about 60% relative humidity for about 2 months. In some embodiments, the composition comprising a compound of formula (I), or a pharmaceutically acceptable salt thereof, comprises no more than about 0.3% area percentage by HPLC of a compound of formula (IV) after storing the composition at about 25° C. and about 60% relative humidity for about 2 months. In some embodiments, the composition comprising a compound of formula (I), or a pharmaceutically acceptable salt thereof, comprises no more than about 0.2% area percentage by HPLC of a compound of formula (IV) after storing the composition at about 25° C. and about 60% relative humidity for about 2 months. In some embodiments, the composition comprising a compound of formula (I), or a pharmaceutically acceptable salt thereof, comprises no more than about 0.1% area percentage by HPLC of a compound of formula (IV) after storing the composition at about 25° C. and about 60% relative humidity for about 2 months.

In some embodiments, the composition comprising a compound of formula (I), or a pharmaceutically acceptable salt thereof, comprises no more than about 0.5% area percentage by HPLC of a compound of formula (IV) after storing the composition at about 25° C. and about 60% relative humidity for about 1 month. In some embodiments, the composition comprising a compound of formula (I), or a pharmaceutically acceptable salt thereof, comprises no more than about 0.4% area percentage by HPLC of a compound of formula (IV) after storing the composition at about 25° C. and about 60% relative humidity for about 1 month. In some embodiments, the composition comprising a compound of formula (I), or a pharmaceutically acceptable salt thereof, comprises no more than about 0.3% area percentage by HPLC of a compound of formula (IV) after storing the composition at about 25° C. and about 60% relative humidity for about 1 month. In some embodiments, the composition comprising a compound of formula (I), or a pharmaceutically acceptable salt thereof, comprises no more than about 0.2% area percentage by HPLC of a compound of formula (IV) after storing the composition at about 25° C. and about 60% relative humidity for about 1 month. In some embodiments, the composition comprising a compound of formula (I), or a pharmaceutically acceptable salt thereof, comprises no more than about 0.1% area percentage by HPLC of a compound of formula (IV) after storing the composition at about 25° C. and about 60% relative humidity for about 1 month.

In some embodiments, the composition comprising a compound of formula (I), or a pharmaceutically acceptable salt thereof, comprises no more than 2% by weight of a compound of formula (II). In some embodiments, the composition comprising a compound of formula (I), or a pharmaceutically acceptable salt thereof, comprises no more than 1.5% by weight of a compound of formula (II). In some embodiments, the composition comprising a compound of formula (I), or a pharmaceutically acceptable salt thereof, comprises no more than 1% by weight of a compound of formula (II). In some embodiments, the composition comprising a compound of formula (I), or a pharmaceutically acceptable salt thereof, comprises no more than 0.5% by weight of a compound of formula (II). In some embodiments, the composition comprising a compound of formula (I), or a pharmaceutically acceptable salt thereof, comprises no more than 0.1% by weight of a compound of formula (II).

In some embodiments, the composition comprising a compound of formula (I), or a pharmaceutically acceptable salt thereof, comprises no more than 2% by weight of a compound of formula (III). In some embodiments, the composition comprising a compound of formula (I), or a pharmaceutically acceptable salt thereof, comprises no more than 1.5% by weight of a compound of formula (III). In some embodiments, the composition comprising a compound of formula (I), or a pharmaceutically acceptable salt thereof, comprises no more than 1% by weight of a compound of formula (III). In some embodiments, the composition comprising a compound of formula (I), or a pharmaceutically acceptable salt thereof, comprises no more than 0.5% by weight of a compound of formula (III). In some embodiments, the composition comprising a compound of formula (I), or a pharmaceutically acceptable salt thereof, comprises no more than 0.1% by weight of a compound of formula (III). In some embodiments, the composition comprising a compound of formula (I), or a pharmaceutically acceptable salt thereof, comprises no more than 0.09% by weight of a compound of formula (III). In some embodiments, the composition comprising a compound of formula (I), or a pharmaceutically acceptable salt thereof, comprises no more than 0.08% by weight of a compound of formula (III). In some embodiments, the composition comprising a compound of formula (I), or a pharmaceutically acceptable salt thereof, comprises no more than 0.07% by weight of a compound of formula (III). In some embodiments, the composition comprising a compound of formula (I), or a pharmaceutically acceptable salt thereof, comprises no more than 0.06% by weight of a compound of formula (III). In some embodiments, the composition comprising a compound of formula (I), or a pharmaceutically acceptable salt thereof, comprises no more than 0.05% by weight of a compound of formula (III). In some embodiments, the composition comprising a compound of formula (I), or a pharmaceutically acceptable salt thereof, comprises no more than 0.04% by weight of a compound of formula (III). In some embodiments, the composition comprising a compound of formula (I), or a pharmaceutically acceptable salt thereof, comprises no more than 0.03% by weight of a compound of formula (III). In some embodiments, the composition comprising a compound of formula (I), or a pharmaceutically acceptable salt thereof, comprises no more than 0.02% by weight of a compound of formula (III). In some embodiments, the composition comprising a compound of formula (I), or a pharmaceutically acceptable salt thereof, comprises no more than 0.01% by weight of a compound of formula (III).

In some embodiments, the composition comprising a compound of formula (I), or a pharmaceutically acceptable salt thereof, comprises no more than 2% by weight of a compound of formula (IV). In some embodiments, the composition comprising a compound of formula (I), or a pharmaceutically acceptable salt thereof, comprises no more than 1.5% by weight of a compound of formula (IV). In some embodiments, the composition comprising a compound of formula (I), or a pharmaceutically acceptable salt thereof, comprises no more than 1% by weight of a compound of formula (IV). In some embodiments, the composition comprising a compound of formula (I), or a pharmaceutically acceptable salt thereof, comprises no more than 0.5% by weight of a compound of formula (IV). In some embodiments, the composition comprising a compound of formula (I), or a pharmaceutically acceptable salt thereof, comprises no more than 0.1% by weight of a compound of formula (IV).

### Methods of Preparation

The compositions described herein are prepared using any method described herein. For example, a composition comprising a compound of formula (I):

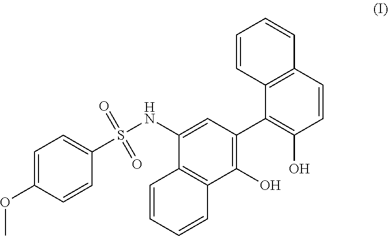 [SMILES: COc1ccc(S(=O)(=O)Nc2cc(-c3c(O)ccc4ccccc34)c(O)c3ccccc23)cc1]

substantially free of an impurity, is prepared by a process comprising:
(a) contacting Compound 2:

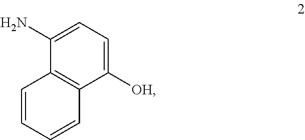 [SMILES: Nc1ccc(O)c2ccccc12]

with Compound 1:

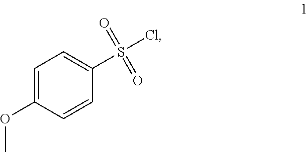

to produce Compound 3:

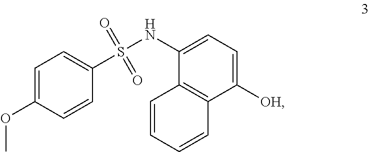 [SMILES: COc1ccc(S(=O)(=O)Nc2ccc(O)c3ccccc23)cc1]

(b) contacting Compound 3 with an oxidizing agent to produce Compound 4:

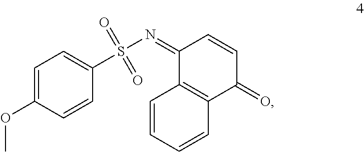 [SMILES: COc1ccc(S(=O)(=O)/N=C2/C=CC(=O)c3ccccc32)cc1]

(c) contacting Compound 4 with Compound 5:

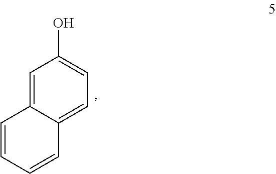 [SMILES: Oc1ccc2ccccc2c1]

in the presence of a coupling agent to produce the compound of formula (I);
(d) contacting the compound of formula (I) of step (c) with an alcohol, water, or a combination thereof, to form a mixture; and
(e) filtering the mixture from step (d) to isolate a precipitate, wherein the precipitate is the composition comprising the compound of formula (I) substantially free of an impurity.

In some embodiments, the oxidizing agent is a periodate salt. In some embodiments, the oxidizing agent is sodium periodate or potassium periodate. In some embodiments, the coupling agent is a boron trifluoride etherate.

In some embodiments, the alcohol is selected from the group consisting of: methanol, ethanol, isopropanol, butanol, propanol, and hexanol, or a mixture thereof. In some embodiments, the alcohol is methanol. In some embodiments, the alcohol is ethanol. In some embodiments, the alcohol is isopropanol. In some embodiments, the alcohol is propanol. In some embodiments, the alcohol is hexanol.

In some embodiments, in step (d), the compound of formula (I) of step (c) is contacted with a combination of alcohol and water to form the mixture. In some embodiments, the alcohol is isopropanol.

In some embodiments, the process further comprises after step (e):
(f) dissolving the precipitate in a first solvent to form a mixture and washing said mixture with a washing solution selected from brine, water, and a combination thereof; and
(g) separating the mixture of step (f) from the washing solution to obtain a separated mixture, and optionally
(h) concentrating the separated mixture of step (g) to form a concentrated mixture.

In some embodiments, the first solvent is selected from 2-methyltetrahydrofuran (2-MeTHF), acetone, dimethyl sulfoxide, and methylethylketone, and mixtures thereof. In some embodiments, the first solvent is selected from 2-MeTHF and acetone. In some embodiments, the first solvent is 2-MeTHF.

In some embodiments, the process further comprises after step (h):
(i) contacting the concentrated mixture with a first solvent and adding charcoal to form a precipitate/charcoal mixture;
(j) filtering the precipitate/charcoal mixture to form a filtrate;
(k) adding a second solvent to the filtrate to form a second mixture comprising a second precipitate; and
(l) filtering the second mixture to retain the second precipitate.

In some embodiments, the process further comprises after step (l), drying the second precipitate.

In some embodiments, the first solvent is selected from 2-methyltetrahydrofuran (2-MeTHF), acetone, dimethyl sulfoxide, and methylethylketone, and mixtures thereof. In some embodiments, the first solvent is selected from 2-MeTHF and acetone. In some embodiments, the first solvent is acetone.

In some embodiments, the second solvent is pentane, hexane, or heptane, or mixtures thereof. In some embodiments, the second solvent is n-heptane.

In some embodiments, the process further comprises after step (e):
(f′) dissolving the compound of formula (I) precipitate in a first solvent and adding charcoal to form a compound of formula (I)/charcoal mixture;
(g′) filtering the compound of formula (I)/charcoal mixture to isolate a filtrate comprising the compound of formula (I) (e.g., wherein the filtering separates the charcoal from the filtrate comprising the compound of formula (I));
(h′) adding a second solvent to the filtrate of step (g′) to form a second mixture comprising a second precipitate comprising the compound of formula (I); and
(i′) filtering the second mixture of step (h′) to isolate the second precipitate comprising the compound of formula (I).

In some embodiments, the process further comprises after step (i′), drying the second precipitate.

In some embodiments, the first solvent is selected from 2-methyltetrahydrofuran (2-MeTHF), acetone, dimethyl sulfoxide, and methylethylketone, and mixtures thereof. In some embodiments, the first solvent is selected from 2-MeTHF and acetone. In some embodiments, the first solvent is acetone. In some embodiments, the first solvent is 2-MeTHF.

In some embodiments, the second solvent is pentane, hexane, or heptane, or mixtures thereof. In some embodiments, the second solvent is n-heptane.

In some embodiments, the impurity is an oxidative product of the compound of formula (I) such as a compound of formula (II). In some embodiments, the impurity is a synthetic impurity such as a compound of formula (III). In some embodiments, the impurity is a compound of formula (IV). In some embodiments, the impurity is selected from the group consisting of: a compound of formula (II), a compound of formula (III), and a compound of formula (IV), or a combination thereof.

In some embodiments, the method produces greater than or equal to 1 kg of the compound of formula (I). In some embodiments, the method produces greater than or equal to 2 kg of the compound of formula (I). In some embodiments, the method produces greater than or equal to 3 kg of the compound of formula (I). In some embodiments, the method produces greater than or equal to 4 kg of the compound of formula (I). In some embodiments, the method produces greater than or equal to 5 kg of the compound of formula (I). In some embodiments, the method produces greater than or equal to 10 kg of the compound of formula (I). In some embodiments, the method produces greater than or equal to 15 kg of the compound of formula (I). In some embodiments, the method produces greater than or equal to 20 kg of the compound of formula (I). In some embodiments, the method produces greater than or equal to 30 kg of the compound of formula (I). In some embodiments, the method produces greater than or equal to 40 kg of the compound of formula (I). In some embodiments, the method produces greater than or equal to 50 kg of the compound of formula (I).

Also disclosed herein is a method of evaluating a composition comprising a compound of formula (I):

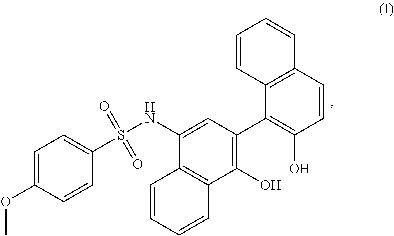 [SMILES: COc1ccc(S(=O)(=O)Nc2cc(-c3c(O)ccc4ccccc34)c(O)c3ccccc23)cc1]

the method comprising assaying the composition for the presence of a compound selected from the group consisting of:

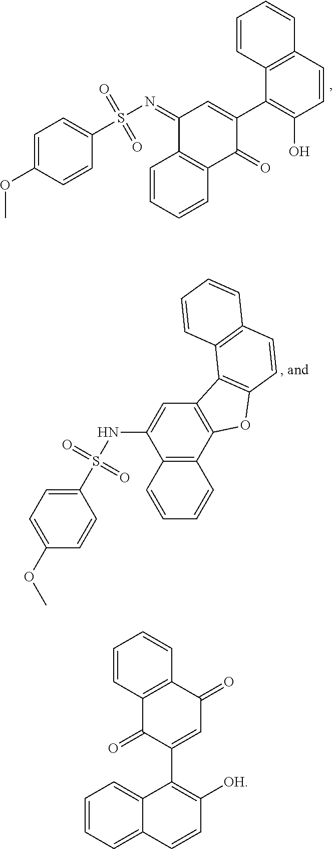 [SMILES: COc1ccc(S(=O)(=O)/N=C2/C=C(c3c(O)ccc4ccccc34)C(=O)c3ccccc32)cc1 ; COc1ccc(S(=O)(=O)Nc2cc3c(oc4ccc5ccccc5c43)c3ccccc23)cc1 ; O=C1C=C(c2c(O)ccc3ccccc23)C(=O)c2ccccc21]

In some embodiments, the assaying comprises analysis using HPLC (high-performance liquid chromatography). In some embodiments, the assaying comprises analysis using MS (mass spectrometry). In some embodiments, the assaying comprises analysis using LC-MS (liquid chromatography-mass spectrometry). In some embodiments, the assaying comprises analysis using NMR (nuclear magnetic resonance). In some embodiments, the assaying comprises analysis using IR (infrared spectroscopy). In some embodiments, the assaying comprises analysis using UV (ultraviolet-visible spectroscopy). In some embodiments, the assay comprises analysis using a combination of HPLC, MS, LC-MS, NMR, IR, or UV.

In some embodiments, the method of evaluating a composition comprising a compound of formula (I) further comprises assaying for the amount of a compound selected from the group consisting of:

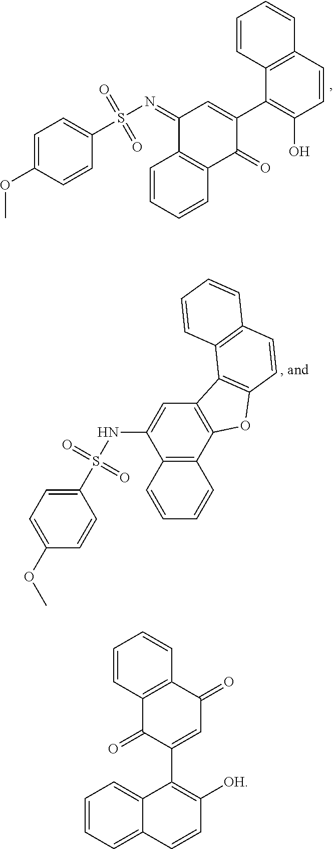 [SMILES: COc1ccc(S(=O)(=O)/N=C2/C=C(c3c(O)ccc4ccccc34)C(=O)c3ccccc32)cc1 ; COc1ccc(S(=O)(=O)Nc2cc3c(oc4ccc5ccccc5c43)c3ccccc23)cc1 ; O=C1C=C(c2c(O)ccc3ccccc23)C(=O)c2ccccc21]

### Pharmaceutical Compositions, Administration, and Dosages

The compositions disclosed herein may further comprise a pharmaceutically acceptable carrier, including, but not limited to, inert solid diluents and fillers, diluents, including sterile aqueous solution and various organic solvents, permeation enhancers, solubilizers and adjuvants. The pharmaceutical compositions may be administered alone or in combination with other therapeutic agents. Such compositions are prepared in any suitable manner (see, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The pharmaceutical compositions may be administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

One mode for administration is parenteral, particularly by injection. The forms in which the compositions of the present disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating a composition according to the present disclosure in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Oral administration is another route for administration of compositions in accordance with the disclosure. Administration may be via capsule or enteric coated tablets, or the like. In making the pharmaceutical compositions that include the compound described herein, the active ingredient is usually diluted by an excipient and/or enclosed within such a carrier that can be in the form of a capsule, sachet, paper or other container. When the excipient serves as a diluent, it can be in the form of a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl and propylhydroxy-benzoates; sweetening agents; and flavoring agents.

The compositions disclosed herein can be formulated so as to provide quick, sustained or delayed release of the active ingredient after administration to the patient by employing procedures known in the art. Controlled release drug delivery systems for oral administration include osmotic pump systems and dissolutional systems containing polymer-coated reservoirs or drug-polymer matrix formulations.

In some embodiments, the compositions are preferably formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The compositions are generally administered in a pharmaceutically effective amount. Preferably, for oral administration, each dosage unit contains from 1 mg to 2 g of a compound described herein, and for parenteral administration, preferably from 0.1 to 1000 mg of a compound a compound described herein. It will be understood, however, that the amount of the compound actually administered usually will be determined by a physician, in the light of the relevant circumstances, including the condition to be treated, the chosen route of administration, the actual compound administered and its relative activity, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, it is meant that the active ingredient is dispersed evenly throughout the composition so that the composition may be readily subdivided into equally effective unit dosage forms such as tablets, pills and capsules.

The tablets or pills of the present disclosure may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably, the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

In some embodiments, compositions comprising a compound of formula (I) are administered to the subject as oral dosage forms. In some embodiments, the oral dosage form is in the form of a tablet. In some embodiments, the oral dosage form is in the form of a capsule.

The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”). Lower or higher doses than those recited above may be required. Specific dosage and treatment regimens for any particular subject will depend upon a variety of factors, including the activity of the specific compound employed, the age, body weight, general health status, sex, diet, time of administration, rate of excretion, drug combination, the severity and course of the disease, condition or symptoms, the subject's disposition to the disease, condition or symptoms, and the judgment of the treating physician. A course of therapy can comprise one or more separate administrations of a compound as described herein.

### Methods of Use

Also provided herein, in certain embodiments, are methods of using compositions comprising a compound of formula (I), or a pharmaceutically acceptable salt thereof, wherein the compositions are substantially free of an impurity, in therapeutic or other methods described herein.

In some embodiments, the methods comprise the administration of a compound of formula (I), a STAT3 inhibitor, wherein the compound of formula (I) is formulated in a manner described herein (e.g., is present in a composition as described herein). In some embodiments, a compound of formula (I) (e.g., as formulated herein e.g., as an oral dosage form) is utilized in a method for treating, preventing, or reducing the risk or severity of a disease or disorder mediated by STAT3, or a disease or disorder that is otherwise treatable with a STAT3 inhibitor. For example, the compositions described herein are useful for treating, preventing, or reducing the risk or severity of certain diseases or disorders characterized by excessive STAT3 protein expression. In specific embodiments, provided herein are methods of treating, preventing, or reducing the risk or severity of cancer. In other specific embodiments, provided herein are methods of treating, preventing, or reducing the risk or severity of fibrosis. In still other specific embodiments, provided herein are methods of treating, preventing, or reducing the risk or severity of an inflammatory disease/disorder.

Signal transducer and activator of transcription 3 (STAT3) is central in regulating the anti-tumor immune response. STAT3 is broadly hyperactivated both in cancer and non-cancerous cells within the tumor ecosystem and plays important roles in inhibiting the expression of crucial immune activation regulators and promoting the production of immunosuppressive factors. Methods provided herein are contemplated as being useful for the treatment of a cancer, including for example, solid tumors, soft tissue tumors, and metastases thereof.

Provided in certain embodiments herein are methods of treating, preventing, or reducing the risk or severity of a cancer in an individual in need thereof, the method comprising administering to the individual any composition described herein. In some embodiments, the cancer treated according to a method provided herein is a liver cancer, lung cancer, head and neck cancer, breast cancer, skin cancer, kidney cancer, testicular cancer, colon cancer, rectal cancer, gastric cancer, skin cancer, metastatic melanoma, prostate cancer, ovarian cancer, cervical cancer, bone cancer, spleen cancer, gall bladder cancer, brain cancer, pancreatic cancer, stomach cancer, anal cancer, prostate cancer, multiple myeloma, post-transplant lymphoproliferative disease, restenosis, myelodysplastic syndrome, leukemia, lymphoma, or acute myelogenous leukemia. In some embodiments, a cancer treated according to a method provided herein is a liver cancer, lung cancer, liver carcinoma, hepatocellular carcinoma, head and neck squamous cell carcinoma, non-small cell lung cancer, or estrogen receptor-positive breast cancer. In some embodiments, a cancer treated according to a method provided herein is head and neck cancer, lung cancer, liver cancer, breast cancer, ovarian cancer, colon cancer, multiple myeloma, leukemia, or pancreatic cancer. In some embodiments, the leukemia is acute myelogenous leukemia.

In some embodiments, provided herein is a method of treating cancer in an individual in need thereof, the method comprising administering to the individual a therapeutically effective amount of the composition disclosed herein. In some embodiments, the cancer is head and neck cancer, lung cancer, liver cancer, breast cancer, skin cancer, testicular cancer, colon cancer, rectal cancer, gastric cancer, bone cancer, spleen cancer, gall bladder cancer, stomach cancer, anal cancer, post-transplant lymphoproliferative disease, restenosis, ovarian cancer, colon cancer, multiple myeloma, prostate cancer, cervical cancer, brain cancer, pancreatic cancer, myelodysplastic syndrome, leukemia, lymphoma, neuroblastoma, kidney cancer, or metastatic melanoma. In some embodiments, the cancer is head and neck cancer, lung cancer, liver cancer, breast cancer, ovarian cancer, colon cancer, multiple myeloma, leukemia, or pancreatic cancer.

Moreover, STAT3 is important for Th17 lymphocyte development and cytokine production, and its activation has been linked to the development of airway inflammation. Upon activation, STAT3 is recruited to cytokine-activated receptor complexes and becomes phosphorylated at Tyr (Y) 705. Phosphotyrosylated (p) STAT3 homodimerizes through reciprocal SH2-pY705 interactions, translocates to the nucleus, and binds to promoters to transcriptionally activate genes that drive Th17 differentiation and production of multiple cytokines. STAT3 activation also is involved in Th2 cytokine production, making it an attractive target for asthma treatment. In addition, several genes have been implicated as risk factors for inflammatory bowel disease (IBD) in genome-wide association studies (GWAS), including ATG16L, NOD2/CARD15, IBD5, CTLA4, TNFSF15, JAK2, STAT3, IL23R, and ORMDL3, which implicate antimicrobial peptides, innate and adaptive immune cell function, Th17 cells, regulatory T cells (Tregs), and cytokines (tumor necrosis factor, interleukins 17, 23, 12, 22, and IL-6). Many of these cytokines serve as ligands for cell surface receptors that activate STAT3. STAT3 within three cell lineages—myeloid cells, enterocytes, and T cells—contribute to colitis in mice and humans. Thus, targeting STAT3 represents an effective means of treating, preventing, or reducing the risk or severity of inflammatory disease/disorder.

Provided in certain embodiments herein are methods of treating, preventing, or reducing the risk or severity of an inflammatory disease/disorder in an individual in need thereof, the method comprising administering to the individual any composition described herein. In some embodiments, the inflammatory disease/disorder treated herein is inflammatory bowel disease (IBD), ulcerative colitis, Crohn's disease, asthma, anaphylaxis, cancer cachexia, chronic kidney disease cachexia, nonalcoholic steatohepatitis (NASH), psoriasis, uveitis, scleritis, multiple sclerosis, or pancreatitis. In some embodiments, inflammation treated herein is inflammatory bowel disease (IBD), ulcerative colitis, Crohn's disease, asthma, anaphylaxis, cancer cachexia, chronic kidney disease cachexia, or nonalcoholic steatohepatitis (NASH). In some embodiments, the anaphylaxis comprises anaphylactic shock.

In some embodiments, provided herein is a method of treating non-alcoholic fatty liver disease or steatohepatitis in an individual in need thereof, the method comprising administering to the individual a therapeutically effective amount of the composition disclosed herein.

In some embodiments, provided herein is a method of treating an inflammatory disease or disorder in an individual in need thereof, the method comprising administering to the individual a therapeutically effective amount of the composition disclosed herein. In some embodiments, the inflammatory disease or disorder is inflammatory bowel disease, ulcerative colitis, psoriasis, uveitis, scleritis, multiple sclerosis, pancreatitis, or asthma.

Fibrosis is a pathological process involving the accumulation of excessive extra-cellular matrix in tissues, leading to tissue damage and organ dysfunction, which can progress to organ failure and death. In systemic sclerosis, an idiopathic fibrosis disease, the trigger is postulated to be an autoimmune response that leads to tissue injury, production of growth factors, pro-inflammatory and pro-fibrotic cytokines, and accumulation of myofibroblasts. Two potential sources of myofibroblasts are the differentiation of local fibroblasts and the process of epithelial-to-mesenchymal transition (EMT). IL-6 is a proinflammatory and profibrotic cytokine increasingly recognized as an important mediator of fibrosis that contribute to the accumulation of myofibroblasts. After engaging its receptor, IL-6 signals through the STAT3. Thus, STAT3 represents a potentially important protein to target to treat fibrosis.

Provided in certain embodiments herein are methods of treating, preventing, or reducing the risk or severity of fibrosis in an individual in need thereof, the method comprising administering to the individual any composition described herein. In certain embodiments, the fibrosis is associated with a disorder or disease such as skin fibrosis (or dermal fibrosis), cardiac fibrosis, cirrhosis, pulmonary fibrosis, bone marrow fibrosis, intestine fibrosis, pancreatic fibrosis, joint fibrosis, liver fibrosis, retroperitoneum, renal fibrosis, myelofibrosis, non-alcoholic fatty liver disease, steatohepatitis, systemic sclerosis (including diffuse systemic sclerosis or limited systemic sclerosis), endomyocardial fibrosis, myocardial infarction, atrial fibrosis, mediastinal fibrosis, progressive massive fibrosis, nephrogenic systemic fibrosis, Keloid, arthrofibrosis, adhesive capsulitis, or cystic fibrosis. In certain embodiments, the fibrosis is associated with skin fibrosis (scleroderma), cardiac fibrosis, cirrhosis, pulmonary fibrosis, bone marrow fibrosis, intestine fibrosis, pancreatic fibrosis, joint fibrosis, liver fibrosis, retroperitoneum, myelofibrosis, non-alcoholic fatty liver disease, steatohepatitis, or systemic sclerosis. In certain embodiments, the fibrosis is associated with skin fibrosis (scleroderma), cardiac fibrosis, cirrhosis, or pulmonary fibrosis.

In some embodiments, provided herein is a method of treating fibrosis in an individual in need thereof, the method comprising administering to the individual a therapeutically effective amount of the composition disclosed herein. In some embodiments, the fibrosis is associated with pulmonary fibrosis, intestine fibrosis, pancreatic fibrosis, joint fibrosis, liver fibrosis, retroperitoneal fibrosis, myelofibrosis, renal fibrosis, bone marrow fibrosis, dermal fibrosis, non-alcoholic fatty liver disease, steatohepatitis, or systemic sclerosis. In some embodiments, the fibrosis is associated with pulmonary fibrosis, non-alcoholic fatty liver disease, steatohepatitis, or systemic sclerosis.

In certain embodiments, the fibrosis is associated with exposure to certain drugs such as chemotherapy, fibrosis following exposure to environmental or other toxins or allergens, fibrosis occurring after an ischemia/reperfusion injury such as myocardial infarction or hypotension, fibrosis occurring after radiation, fibrosis following hepatitis induced by alcohol, toxins, drugs or infections, primary biliary cirrhosis, fibrosis following viral infections involving the heart, liver, or lung, and/or idiopathic retroperitoneal fibrosis.

Muscle wasting is a debilitating complication of catabolic conditions including chronic kidney disease (CKD), diabetes, cancer, or serious infections. For example, in mice with CKD, inhibition of myostatin reduced circulating levels of IL-6 and TNFα, suggesting a link between inflammation and muscle wasting as reported in clinical studies. STAT3 was found to be activated by the IL-6 family of cytokines, thus suggesting that the STAT3 pathway is linked to loss of muscle mass.

Provided in certain embodiments herein are methods of treating, preventing, or reducing the risk or severity of a muscle wasting disease/disorder, muscle weakness disease/disorder, or cachexia in an individual in need thereof, the method comprising administering to the individual any composition described herein. The muscle weakness and/or muscle wasting and/or cachexia may have an unknown cause or it may be associated with an underlying condition. The underlying condition may be a catabolic condition. In some embodiments, the underlying medical condition associated with cachexia is least renal disease or failure, cancer, AIDS, HIV infection, chronic obstructive lung disease (including emphysema), multiple sclerosis, congestive heart failure, tuberculosis, familial amyloid polyneuropathy, acrodynia, hormonal deficiency, metabolic acidosis, infectious disease, chronic pancreatitis, autoimmune disorder, celiac disease, Crohn's disease, electrolyte imbalance, Addison's disease, sepsis, burns, trauma, fever, long bone fracture, hyperthyroidism, prolonged steroid therapy, surgery, bone marrow transplant, atypical pneumonia, brucellosis, endocarditis, Hepatitis B, lung abscess, mastocytosis, paraneoplastic syndrome, polyarteritis nodosa, sarcoidosis, systemic lupus erythematosus, myositis, polymyositis, dematomyosytis, rheumatological diseases, autoimmune disease, collagen-vascular disease, visceral leishmaniasis, prolonged bed rest, and/or addiction to drugs, such as amphetamine, opiates, or barbitutates.

In some embodiments, provided herein is a method of treating cachexia in an individual in need thereof, the method comprising administering to the individual a therapeutically effective amount of the composition disclosed herein.

In addition, STAT3 signaling has been implicated in gap junction intercellular communication, IL-6- and IL11-induced vascular leakage, down-regulation of VE-cadherin concomitant with phosphorylation of STAT3, and the STAT3/mir17-92/E2F1 dependent regulation of β-catenin nuclear translocation and transcriptional activity. Thus, STAT3 inhibition is useful to reduce vascular permeability in the setting of anaphylaxis.

In some embodiments, provided herein is a method of treating anaphylaxis in an individual in need thereof, the method comprising administering to the individual a therapeutically effective amount of the composition disclosed herein.

Provided in certain embodiments herein are methods of treating, preventing, or reducing the risk or severity of an allergic reaction in an individual in need thereof, the method comprising administering to the individual any composition described herein. In some embodiments, the allergic reaction is induced following an exposure to an allergen. In some embodiments, the allergen is a food allergen (such as milk, legumes, shellfish, tree nuts, eggs, fish, soy, and wheat), an environmental allergen or seasonal allergen (such as pollen or mold), a venom allergen (such as from wasp, bee, ant, hornet, yellow jacket, or asp), a medication allergen (such as anesthetics, β-lactam antibiotics, aspirin, non-steroidal anti-inflammatory drug, chemotherapy, vaccine, protamine, or herbal preparations), or latex. In some embodiments, the allergic reaction is anaphylaxis, anaphylactic shock, allergic rhinitis, urticaria, food allergy, drug allergy, hymenoptera allerga, bronchial constriction, asthma, or eczema.

STAT3 also plays an important role in viral infection and pathogenesis. Provided in certain embodiments herein are methods of treating, preventing, or reducing the risk or severity of a viral infection in an individual in need thereof, the method comprising administering to the individual any composition described herein. In some embodiments, the viral infection is a chronic viral infection. In some embodiments, the chronic viral infection is AIDS, HIV infection, Hepatitis B infection, Hepatitis C virus infection, or Epstein-Barr virus infection.

In addition, reactive astrocytes in neurodegenerative diseases including Alzheimer's disease are implicated in STAT3 phosphorylation. Pathophysiological roles of astrocytes in the reactive state are thought to have important significance in the pathogenesis of neurodegenerative diseases. Provided in certain embodiments herein are methods of treating, preventing, or reducing the risk or severity of a neurodegenerative disease in an individual in need thereof, the method comprising administering to the individual any composition described herein. In some embodiments, the neurodegenerative disease is chemotherapy-induced peripheral neuropathy, diabetic neuropathy, or chemobrain. Provided in certain embodiments herein are methods of treating, preventing, or reducing the risk or severity of pain in an individual in need thereof, the method comprising administering to the individual any composition described herein. In some embodiments, pain is neuropathic pain. Provided in certain embodiments herein are methods of treating, preventing, or reducing the risk or severity of graft-versus-host diseases, pulmonary lymphangioleiomyomatosis, chagasic cardiomyopathy, age-related macular degeneration, amyloidosis, astrogliosis in Alzheimer's or other neurodegenerative diseases, or familial amyloid polyneuropathy.

In some embodiments, provided herein is a method of treating a neurodegenerative disease or disorder in an individual in need thereof, the method comprising administering to the individual a therapeutically effective amount of the composition disclosed herein. In some embodiments, provided herein is a method of treating chemotherapy-induced peripheral neuropathy in an individual in need thereof, the method comprising administering to the individual a therapeutically effective amount of the composition disclosed herein. In some embodiments, provided herein is a method of treating diabetic neuropathy in an individual in need thereof, the method comprising administering to the individual a therapeutically effective amount of the composition disclosed herein. In some embodiments, provided herein is a method of treating familial amyloid polyneuropathy in an individual in need thereof, the method comprising administering to the individual a therapeutically effective amount of the composition disclosed herein.

STAT3 is involved in cytokine- and nutrient-induced insulin resistance, and excessive STAT3 signaling is implicated in the development of insulin resistance such as skeletal muscle insulin resistance in type 2 diabetes. Provided in certain embodiments herein are methods of treating, preventing, or reducing the risk or severity of insulin resistance in an individual in need thereof, the method comprising administering to the individual any composition described herein. In some embodiments, the insulin resistance is a result of an underlying condition. In some embodiments, the insulin resistance is associated with muscle of the individual being treated. In some embodiments, the insulin resistance is caused by any reason for the individual, such as elevated free fatty acids in the blood, obesity, being overweight, having visceral fat, having a high fructose intake, having inflammation, being inactive, dysbiosis of the gut microbiota, and/or being genetically predisposed. In certain embodiments, any method provided herein is a method of treating, preventing, or reducing the risk or severity of medical conditions associated with insulin resistance or that are complications of insulin resistance at least in part, such as severe high blood sugar; severe low blood sugar; heart attack; stroke; kidney disease (including chronic, for example, chronic kidney disease (CKD)); eye problems; cancer; non-alcoholic fatty liver disease (NAFLD); polycystic ovarian syndrome (PCOS); metabolic syndrome; diabetes; or Alzheimer's disease, for example. In certain embodiments, the insulin resistance is a hallmark of metabolic syndrome and type 2 diabetes. Metabolic syndrome is a group of risk factors associated with type 2 diabetes and heart disease. Its symptoms include high blood triglycerides, blood pressure, belly fat, and blood sugar, as well as low HDL (good) cholesterol levels.

In some embodiments, provided herein is a method of treating non-alcoholic fatty liver disease or steatohepatitis in an individual in need thereof, the method comprising administering to the individual a therapeutically effective amount of the composition disclosed herein.

In some embodiments, the methods comprise administering a therapeutically effective amount of a composition disclosed herein to the individual. In certain embodiments, the method comprises administering at least 1 mg/kg/day of the compound of formula (I) to the individual. In certain embodiments, the method comprises administering at least 10 mg/kg/day of the compound of formula (I) to the individual. In certain embodiments, the method comprises administering at least 20 mg/kg/day of the compound of formula (I) to the individual. In certain embodiments, the method comprises administering at least 25 mg/kg/day of the compound of formula (I) to the individual.

### EXAMPLES

Exemplary HPLC conditions: diluent was 0.1% trifluoroacetic acid (TFA) in acetonitrile, Mobile Phase A was 0.05% TFA in water, and Mobile Phase B was 0.05% TFA in acetonitrile, column (Agilent, Eclipse XDB-C18, 3.0×100 mm, particle size 1.8 μm), flow (0.425 mL/min), and detector (280 nm diode array).

### Example 1. Synthesis of TTI-101

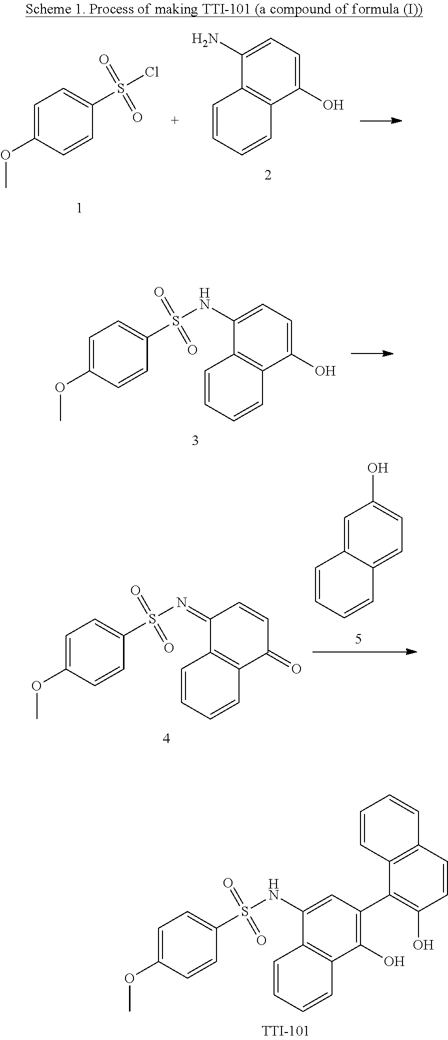 [SMILES: COc1ccc(S(=O)(=O)Cl)cc1 ; Nc1ccc(O)c2ccccc12 ; COc1ccc(S(=O)(=O)Nc2ccc(O)c3ccccc23)cc1 ; COc1ccc(S(=O)(=O)/N=C2/C=CC(=O)c3ccccc32)cc1 ; Oc1ccc2ccccc2c1 ; COc1ccc(S(=O)(=O)Nc2cc(-c3c(O)ccc4ccccc34)c(O)c3ccccc23)cc1]

### Method 1

A mixture of 4-aminonaphthalen-1-ol HCL salt (281 g, 1.44 mol), sodium acetate (358 g, 4.35 mol), 4-methoxybenzene sulfonyl chloride (330 g, 1.60 mol) and purified water (5.4 L) was stirred and heated at 80° C. for about 5 hours. After cooling the mixture to room temperature, the resulting solid was collected by filtration and washed with water. The solid was dried to afford Compound 3 (466 g, 98.5% yield).

To a solution of sodium periodate (105 g) in water (466 g) was added dichloromethane (DCM) (7 L) and silica gel (830 g) and the mixture was stirred. Compound 3 (451 g) in DCM (1.4 L) was added to the mixture and stirred for 6 hours. Then, sodium sulfate (707 g) was added to the mixture and stirred for 2 hours. The mixture was filtered and rinsed with DCM. To this mixture, 2-naphthol (200 g) and boron trifluoride diethyl etherate (8 mL, 0.06 mol) were added and stirred for about 0.5 hour at 38° C. Additional boron trifluoride diethyl etherate (8 mL, 0.06 mol) was added to the mixture and stirred for 2.5 hours at 38° C. The reaction mixture was cooled to room temperature, filtered, and dried under reduced pressure to afford TTI-101 (616 g, 95.3% yield, 99.5% purity by HPLC). The collected solid was dissolved in acetone (4.5 L). Activated carbon (123 g) was added to the solution and stirred for about 2 hours and filtered over Celite®. Then, n-heptane (8 L) was added to the combined filtrate, stirred, and allowed to sit overnight to afford a slurry. The slurry was filtered and the solid was dried under pressure for 18 hours to afford TTI-101 (556 g, 86.0% overall yield, 99.8214% purity by HPLC). HPLC analysis also showed Compound 6 at 0.1111% AUC and Compound 7 at about 0.02% AUC:

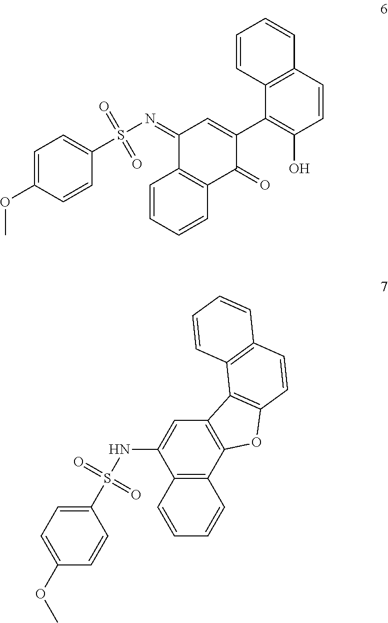 [SMILES: COc1ccc(S(=O)(=O)/N=C2/C=C(c3c(O)ccc4ccccc34)C(=O)c3ccccc32)cc1 ; COc1ccc(S(=O)(=O)Nc2cc3c(oc4ccc5ccccc5c43)c3ccccc23)cc1]

Method 1-a: TTI-101 (10.03 g) obtained from Method 1 described above was combined with acetone (40 mL) with stirring under nitrogen. More acetone was added (33 mL) to the mixture with heating to 53° C. by mantle and stirred to dissolve the solids. Heptanes (55 mL) were added in portions with stirring, and the resulting slurry was cooled to 22° C. then further chilled at 4-5° C. The slurry was filtered under reduced pressure through a funnel, with chilled solvent (1:1 acetone to heptanes) to complete the transfer of solids to the funnel and wash the solids. The solids were dried under reduced pressure with no heating. The purity of the isolated solid (80.8% recovery) was determined by HPLC to be 99.8445% AUC with Compound 6 at 0.1047% AUC.

Method 1-b: TTI-101 (9.82 g) obtained from Method 1 described above was combined with 1:1 acetone and heptanes (100 mL) with stirring under nitrogen at ambient temperature then cooled in an ice/water bath. The resulting slurry was filtered through a funnel under reduced pressure, with chilled solvent (1:1 acetone to heptanes) to complete the transfer of solids to the funnel and wash the solids. The solids were dried under reduced pressure with no heating. The purity of the isolated solid (82.5% recovery) was determined by HPLC to be 99.8404% AUC with Compound 6 at 0.0982% AUC.

### Method 2

A 5 kg batch of TTI-101 was synthesized using the analogous synthetic steps described in Method 1. HPLC analysis showed that the API product contained about 2% AUC of Compound 7. The batch also contained Compound 6 prior to recrystallization and/or slurrying methods in Table 1.

Table 1 describes exemplary recrystallization and/or slurrying of TTI-101 from the batch obtained according to Method 2.

TABLE 1

| TTI-101 | | | | Recovery |
|---|---|---|---|---|
| (g) | Solvent | Conditions | HPLC % purity | yield (%) |
| 1 | Acetone/isopropyl | TTI-101 was dissolved in 10 mL | TTI-101: 98.9% | 94 |
| | alcohol | acetone and 10 mL IPA. The | Cmp 6: 0.33% | |
| | (IPA) | solvents were reduced to ~½ | Cmp 7: 0.78% | |
| | | volume, and the mixture was cooled | | |
| | | to ≤5° C. The solid was filtered and | | |
| | | dried at about 45° C. | | |
| 4 | Acetone/IPA | TTI-101 in IPA (25 mL) was heated | TTI-101: 98.9% | 88 |
| | | to reflux and added additional IPA | Cmp 6: 0.32% | |
| | | (125 mL) and 40 mL acetone. The | Cmp 7: 0.78% | |
| | | solvents were reduced to ~½ | | |
| | | volume, and the mixture was cooled | | |
| | | to ≤5° C. The solid was filtered and | | |
| | | dried at about 45° C. | | |
| 2 | Acetone/heptanes | TTI-101 in acetone (20 mL) was | TTI-101: 99.4% | 85 |
| | | heated to about 50° C. and added 0.2 g | Cmp 6: 0.26% | |
| | | DarcoG60 and stirred. The mixture | Cmp 7: 0.30% | |
| | | was filtered. To the filtrate, heptanes | | |
| | | (25 mL) was added slowly and the | | |
| | | resulting mixture was left to stand | | |
| | | overnight, then cooled to ≤ 5° C. The | | |
| | | solid was filtered and dried at about | | |
| | | 45° C. | | |
| 2 | IPA/acetone | TTI-101 in IPA (25 mL) was heated | TTI-101: 99.4% | 93 |
| | | to 70-80° C. and added about 15 mL | Cmp 6: 0.26% | |
| | | acetone. The solution was cooled to | Cmp 7: 0.30% | |
| | | RT and stirred overnight, then | | |
| | | filtered, and the filtrate was | | |
| | | evaporated to ~½ volume. The | | |
| | | mixture was cooled to ≤ 5° C. The | | |
| | | solid was filtered and dried at about | | |
| | | 45° C. | | |
| 2 | IPA/acetone | TTI-101 in acetone (25 mL) at about | TTI-101: 99.2% | 98 |
| | | 25° C. was filtered. To the filtrate, 50 | Cmp 6: 0.27% | |
| | | mL IPA was slowly added. The | Cmp 7: 0.50% | |
| | | solution was stirred overnight. The | | |
| | | solution was evaporated to ~½ | | |
| | | volume, cooled to ≤ 5° C. The solid | | |
| | | was filtered and dried at about 45° C. | | |
| 5 | Acetone | TTI-101 was slurried with acetone | TTI-101: 98.6% | 49 |
| | | (15 mL) at ambient temperature, | Cmp 6: 0.15% | |
| | | filtered, rinsed with 1:1 | Cmp 7: 1.27% | |
| | | acetone/heptanes (15 mL) and dried | | |
| | | under reduced pressure at 46° C. | | |
| 5 | Acetone | TTI-101 was slurried with acetone | TTI-101: 98.7% | 59 |
| | | (15 mL) at 50° C. then at ambient | Cmp 6: 0.10% | |
| | | temperature, filtered, rinsed with 1:1 | Cmp 7: 1.17% | |
| | | acetone/heptanes (15 mL) and dried | | |
| | | under reduced pressure at 46° C. | | |
| 5 | Acetone/heptanes | TTI-101 was slurried with 1:1 | TTI-101: 99.1% | 83 |
| | | acetone/heptanes (50 mL) at | Cmp 6: 0.06% | |
| | | ambient temperature, filtered, rinsed | Cmp 7: 0.87% | |
| | | with 1:1 acetone/heptanes (15 mL) | | |
| | | and dried under reduced pressure at | | |
| | | 47° C. | | |
| 5 | Acetone/heptanes | TTI-101 in acetone (60 mL) was | TTI-101: 99.2% | 89 |
| | | stirred at 50° C. then added heptanes | Cmp 6: 0.07% | |
| | | (60 mL). The mixture was cooled | Cmp 7: 0.75% | |
| | | slowly to ambient temperature and | | |
| | | stirred overnight. The slurry was | | |
| | | filtered, rinsed with 1:1 | | |
| | | acetone/heptanes (15 mL) and dried | | |
| | | under reduced pressure at 47° C. | | |
| 3 | Ethanol | TTI-101 was slurried with EtOH | TTI-101: 98.4% | 76 |
| | | (109 mL) at 75° C., cooled and then | Cmp 6: 0.44% | |
| | | slurried at 45° C. The slurry was | Cmp 7: 0.81% | |
| | | filtered, rinsed with ethanol (6 mL) | | |
| | | and dried overnight. | | |
| 0.52 | Acetone | TTI-101 was partially dissolved in | TTI-101: 99.1% | N/A |
| | | acetone, heated at 45° C. surface | Cmp 6: 0.16% | |
| | | temperature, gravity filtered, and | Cmp 7: 0.70% | |
| | | concentrated to about ~0.5 mL to a | | |
| | | waxy solid. | | |
| 4 | Acetone/water | To a mixture of TTI-101 in acetone | TTI-101: 98.2% | 91 |
| | | (50 mL) at 50° C. was added water | Cmp 6: 0.45% | |
| | | (32 mL). The mixture was stirred and | Cmp 7: 0.92% | |
| | | cooled slowly to ambient | | |
| | | temperature and set still overnight. | | |
| | | The slurry was filtered, rinsed with | | |
| | | 6:4 acetone/water (10 mL), dried | | |
| | | under reduced pressure at 47° C. | | |
| 4.1 | Acetone/EtOH | To a mixture of TTI-101 in acetone | TTI-101: 28.1% | 2 |
| | | (50 mL) at 50° C. was added EtOH | Cmp 6: 0.72% | |
| | | (60 mL). The mixture was stirred and | Cmp 7: 70.7% | |
| | | cooled slowly to ambient | | |
| | | temperature and stirred overnight. | | |
| | | The mixture was cooled in ice/water | | |
| | | bath for about 5 hours, placed in the | | |
| | | refrigerator for 4 days, filtered, dried | | |
| | | under reduced pressure at 49° C. | | |
| 4.1 | Acetone/EtOH | To a mixture of TTI-101 in acetone | TTI-101: 99.7% | 50 |
| | | (50 mL) at 50° C. was added EtOH | Cmp 6: 0.16% | |
| | | (60 mL). The mixture was stirred and | Cmp 7: 0.15% | |
| | | cooled slowly to ambient | | |
| | | temperature and stirred overnight. | | |
| | | The mixture was cooled in ice/water | | |
| | | bath for about 5 hours, placed in the | | |
| | | refrigerator for 4 days, filtered. The | | |
| | | filtrate sat overnight to form solids. | | |
| | | The solids were filtered, rinsed with | | |
| | | 1:1 acetone/EtOH (10 mL), and dried | | |
| | | under reduced pressure at 43° C. | | |
| 0.1 | DCM | TTI-101 was slurried with DCM (5 | TTI-101: 99.3% | N/A |
| | | mL), filtered, rinsed with DCM (3-4 | Cmp 6: 0.31% | |
| | | mL) and dried under reduced | Cmp 7: 0.39% | |
| | | pressure at 49° C. | | |
| 0.1 | MeCN | TTI-101 was slurried with warm | TTI-101: 98.7% | N/A |
| | | MeCN (5 mL), filtered, rinsed with | Cmp 6: 0.30% | |
| | | MeCN (about 3 mL) and dried under | Cmp 7: 0.98% | |
| | | reduced pressure at 50° C. | | |
| 1.0 | Acetone/IPA | TTI-101 was dissolved in 10 mL | TTI-101: 98.9% | 94 |
| | | acetone and 10 mL IPA. The | Cmp 6: 0.33% | |
| | | solvents were reduced to ~½ | Cmp 7: 0.78% | |
| | | volume, and cooled to ≤5° C. The | | |
| | | solid was filtered and dried at about | | |
| | | 45° C. | | |
| 4.0 | Acetone/IPA | TTI-101 in IPA (25 mL) was heated | TTI-101: 98.9% | 88 |
| | | to reflux and added additional IPA | Cmp 6: 0.32% | |
| | | (125 mL). To the warm mixture was | Cmp 7: 0.78% | |
| | | added 40 mL acetone. The solvents | | |
| | | were reduced to ~½ volume, and | | |
| | | cooled to ≤5° C. The solid was | | |
| | | filtered and dried at about 45° C. | | |
| 2.0 | Acetone/heptanes | TTI-101 in acetone (20 mL) was | TTI-101: 99.4% | 85 |
| | | heated to about 50° C. and added 0.2 g | Cmp 6: 0.26% | |
| | | DarcoG60 and stirred. The mixture | Cmp 7: 0.30% | |
| | | was filtered. To the filtrate, heptanes | | |
| | | (25 mL) was added slowly and the | | |
| | | resulting mixture was left to stand | | |
| | | overnight, then cooled to ≤5° C. The | | |
| | | solid was filtered and dried at about | | |
| | | 45° C. | | |
| 2.0 | IPA/acetone | TTI-101 in IPA (25 mL) was heated | TTI-101: 99.4% | 93 |
| | | to 70-80° C. and added about 15 mL | Cmp 6: 0.26% | |
| | | acetone. The solution was cooled to | Cmp 7: 0.30% | |
| | | RT and stirred overnight, then | | |
| | | filtered, and the filtrate was | | |
| | | evaporated to ~½ volume. The | | |
| | | mixture was cooled to ≤5° C. The | | |
| | | solid was filtered and dried at about | | |
| | | 45° C. | | |
| 2.0 | IPA/acetone | TTI-101 in IPA (25 mL) was heated | TTI-101: 99.2% | 98 |
| | | to 70-80° C. and added about 15 mL | Cmp 6: 0.27% | |
| | | acetone. The solution was cooled to | Cmp 7: 0.50% | |
| | | RT and stirred overnight, then | | |
| | | filtered, and the filtrate was | | |
| | | evaporated to ~½ volume. The | | |
| | | mixture was cooled to ≤5° C. The | | |
| | | solid was filtered and dried at about | | |
| | | 45° C. | | |

### Method 3

A mixture of 4-aminonaphthalen-1-ol HCL salt (5.0 kg, 25.6 mol), potassium acetate (7.5 kg, 76.8 mol), 4-methoxybenzene sulfonyl chloride (5.8 kg, 28.2 mol) and purified water (75 kg) was agitated and heated at 80° C. for 2 hours. After cooling the mixture to room temperature, the resulting solid was collected by filtration and washed with water. The solid was dried to afford Compound 3 (7.79 kg, 92.7% yield).

A solution of sodium periodate (1.9 kg) in water (34 kg) was added to a mixture of Compound 3 (7.0 kg) in DCM (139 kg). The resulting mixture was agitated at 20° C. for 1 hour and held at 25° C. for 3 hours. The layers were separated and the organic layer was washed twice with brine. The mixture was twice vacuum distilled and diluted with DCM (70 L) then distilled to a final volume of 70 L. To this mixture, 2-naphthol (3.4 kg) and a solution of boron trifluoride dibutyl etherate (40.19 g) in DCM (10 L) were added and agitated for 6.5 hours. The reaction mixture was quenched with 70% wet IPA (7 L). The resulting solid was collected, dissolved in 2-MeTHF (105 L) and washed with 10% brine (2×35 L) and water (1×35 L). The mixture was concentrated and charged with acetone. The resulting mixture was charged onto activated charcoal and agitated for 12 hours. The mixture was filtered over Celite® (Diatomaceous earth) and concentrated. N-heptane was added to the mixture and agitated for 2 hours at 45° C., cooled to 25° C., and agitated for 12 hours. The resulting solid was filtered, washed with a mixture of acetone/n-heptane then with n-heptane, then dried to afford TTI-101 (6.46 kg, 64.6% yield, 99.95% purity). No formation of Compound 7 was detected.

Another batch of TTI-101 was prepared according to the above Method 3 to yield 17.5 kg of TTI-101. No formation of Compound 7 was detected. Purity of TTI-101 as determined by HPLC was on average 99.8%, with 0.13% AUC of Compound 6.

Another batch of TTI-101 was prepared according to the above Method 3 to yield 40.0 kg of TTI-101. No formation of Compound 7 was detected. Purity of TTI-101 as determined by HPLC was on average 100.0%, with 0.05% AUC of Compound 6.

Another batch of TTI-101 was prepared according to the above Method 3 to yield 17.4 kg of TTI-101. No formation of Compound 7 was detected. Purity of TTI-101 as determined by HPLC was on average 99.9%, with 0.09% AUC of Compound 6.

### Method 4

The batch of TTI-101 (6.4 kg) from Method 3 was dissolved in 2-MeTHF, warmed up to 50° C. then cooled to 35° C., transferred to a reactor containing n-heptane through an in-line filter (0.45 micron) over 4 hours. The resulting slurry was adjusted to 25± 5° C. and agitated at 25±5° C. for 12 hours. The slurry was filtered and the solids washed with n-heptane (21.9 kg). The solids were dried and delumped to give TTI-101 (5.89 kg, 92.0% yield, 99.76% purity). No formation of Compound 7 was detected.

### Method 5

A mixture of 4-aminonaphthalen-1-ol HCL salt (5.0 kg, 25.6 mol), potassium acetate (7.5 kg, 76.8 mol), 4-methoxybenzene sulfonyl chloride (5.8 kg, 28.2 mol) and purified water (75 kg) was agitated and heated at 80° C. for 2 hours. After cooling the mixture to room temperature, the resulting solid was collected by filtration and washed with water. The solid was dried to afford Compound 3 (7.78 kg, 92.6% yield).

A solution of sodium periodate (1.8 kg) in water (34 kg) was added to a mixture of Compound 3 (7.0 kg) in DCM (139 kg). The resulting mixture was agitated at 20° C. for 1 hour and held at 25° C. for 3 hours. The reaction mixture was filtered through Celite® and washed with DCM. The layers were separated and the organic layer was washed twice with brine. The mixture was twice vacuum distilled and diluted with DCM (70 L) then distilled to a final volume of 70 L. To this mixture, 2-naphthol (3.4 kg) and a solution of boron trifluoride dibutyl etherate (61.0 g) in DCM (10 L) were added and agitated for 6 hours. The reaction mixture was quenched with 70% wet IPA (7 L). The resulting solid was collected, washed with DCM, and dried. The solid was dissolved in 2-MeTHF (105 L) and washed with 10% brine (2×35 L) and water (1×35 L). The resulting mixture was charged onto activated charcoal and agitated for 12 hours. The mixture was filtered over Celite® and concentrated. The filtrate was added to n-heptane and agitated for 12 hours at 25° C. The resulting solid was filtered, washed with n-heptane, then dried to afford TTI-101 (5.85 kg, 58.5% yield, 99.42% purity). No formation of Compound 7 was detected.

### Example 2. Synthesis of Compound 7

### Method A.

TTI-101 (0.52 g) and boron trifluoride etherate (0.13 mL) was stirred in DCM (25 mL) under nitrogen for about 16.5 hours at 38° C. and at ambient temperature for about 3 days. The resulting slurry was filtered under reduced pressure and washed with DCM. The solid was dried under reduced pressure at 42° C. HPLC analysis showed 1.93% conversion to Compound 7.

### Method B.

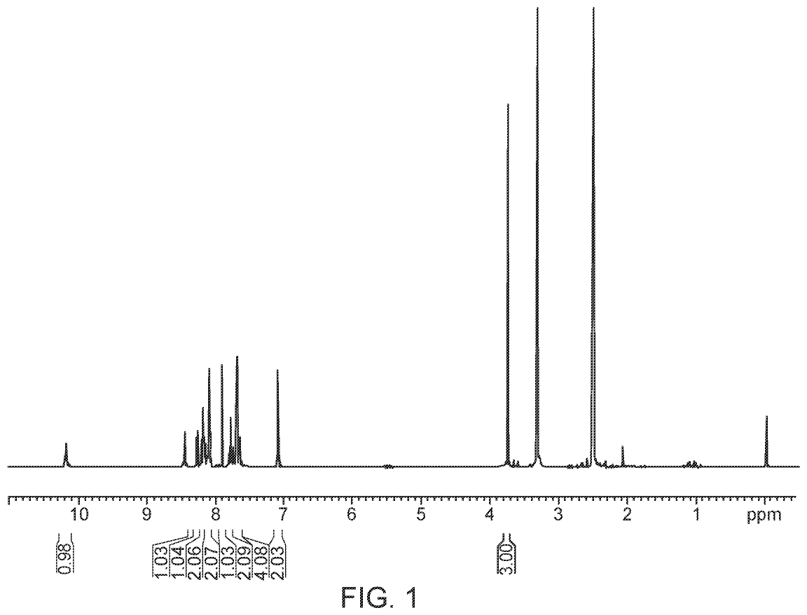

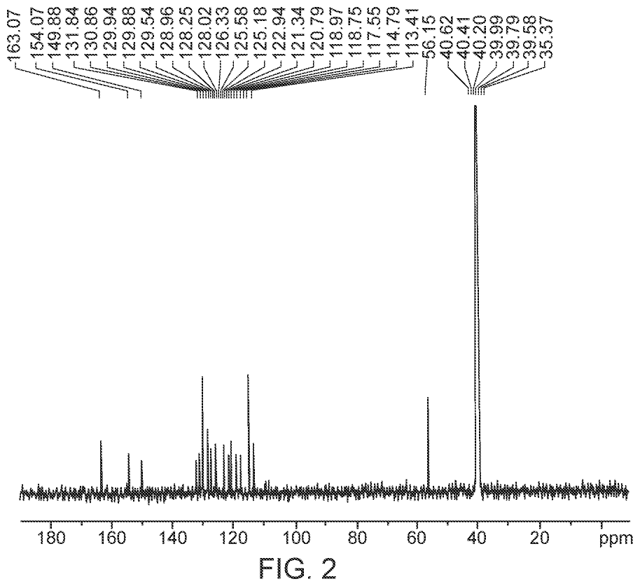

TTI-101 (0.51 g) and boron trifluoride etherate (0.33 mL) was stirred in acetonitrile (25 mL) under nitrogen for about 4 days at 50° C. with additional boron trifluoride etherate (0.20 mL) added at the 30 hour point. The resulting slurry was cooled to ambient temperature, filtered under reduced pressure and washed with acetonitrile. The solid was dried under reduced pressure at 50° C.-55° C. HPLC analysis showed Compound 7 at 99.87% AUC purity. 1H-NMR and 13C-NMR of the product are shown in FIG. 1 and FIG. 2, respectively.

### Example 3. Accelerated Stability and Low Temperature Stability Study of TTI-101

Batch A was prepared analogously using Method 3 as described in Example 1. Batch B was prepared according to Method 5 as described in Example 1.

### Long Term Stability for Batch A

Samples of TTI-101 were stored at 5° C., 25° C./60% RH, 30° C./65% RH, or 40° C./75% RH. TTI-101 was stored in an LDPE bag enclosed with a Nylon cable tie and then transferred to another LDPE bag and enclosed with a Nylon cable tie. The enclosure was put into an HDPE bottle and closed with a polyethylene-lined polypropylene cap. The samples were stored under the above-identified conditions.

### Long Term Stability for Batch B

Samples of TTI-101 were stored at 5° C., 25° C./60% RH, 30° C./65% RH, or 40° C./75% RH. TTI-101 was stored in an LDPE bag enclosed with a Nylon cable tie and then transferred to another LDPE bag and enclosed with a Nylon cable tie. The enclosure was put into an HDPE bottle and closed with a polyethylene-lined polypropylene cap. The samples were stored under the above-identified conditions.

The samples were analyzed using HPLC with the exemplary conditions identified above. Relative retention time (RRT) for Compound 5 was about 0.83, Compound 4 was about 1.05-1.06, Compound 6 was about 1.15-1.17, and compound of formula (IV) was about 0.99.

### ABBREVIATIONS

NA not applicable
NQ ≥0.02% and <0.05%
Avg average
Cmpd compound
— no peak
M month
RRT relative retention time

### For Batch A:

a) Storage condition: 5° C.:

| | Test | | | | | | | | | | | |
|---|---|---|---|---|---|---|---|---|---|---|---|---|
| | 0 M | | | | 2 M | | | | 3 M | | | |
| | Prep | | | | | | | | | | | |
| | | P1 | P2 | Avg | | P1 | P2 | Avg | | P1 | P2 | Avg |
| | Assay (%) | | | | | | | | | | | |
| | | 99.9 | 99.7 | 99.8 | | 99.7 | 100.7 | 100.2 | | 100.2 | 99.5 | 99.9 |
| | Purity (%) | | | | | | | | | | | |
| | | 99.8 | 99.8 | 99.8 | | 99.9 | 100.0 | 99.9 | | 99.7 | 99.7 | 99.7 |
| | Impurities | | | | | | | | | | | |
| | | P1 | P2 | Avg | | P1 | P2 | Avg | | P1 | P2 | Avg |
| | RRT | (area %) | (area %) | (area %) | RRT | (area %) | (area %) | (area %) | RRT | (area %) | (area %) | (area %) |
| | 0.99 | NQ | — | NQ | 0.99 | NQ | — | NQ | — | — | — | — |
| | 1.06 | — | NQ | NQ | — | — | — | — | 1.06 | 0.06 | 0.07 | 0.07 |
| | (Cmpd | | | | | | | | (Cmpd | | | |
| | 4) | | | | | | | | 4) | | | |
| | 1.17 | 0.13 | 0.12 | 0.13 | 1.16 | 0.08 | NQ | 0.08 | 1.16 | 0.22 | 0.22 | 0.22 |
| | (Cmpd | | | | (Cmpd | | | | (Cmpd | | | |
| | 6) | | | | 6) | | | | 6) | | | |
| | 1.32 | NQ | NQ | NQ | — | — | — | — | — | — | — | — |
| | Total | 0.1 | 0.1 | 0.1 | Total | 0.1 | NQ | 0.1 | Total | 0.3 | 0.3 | 0.3 |
| Water | | <0.04 | <0.04 | <0.04 | | <0.04 | <0.04 | <0.04 | | <0.04 | <0.04 | <0.04 |
| Content (%) | | | | | | | | | | | | |
| | Test | | | | | | | | | | | |
| | 6 M | | | | 9 M | | | | 12 M | | | |
| | Prep | | | | | | | | | | | |
| | | P1 | P2 | Avg | | P1 | P2 | Avg | | P1 | P2 | Avg |
| | Assay (%) | | | | | | | | | | | |
| | | 100.6 | 98.3 | 99.4 | | 99.6 | 99.6 | 99.6 | | 99.1 | 99.2 | 99.2 |
| | Purity (%) | | | | | | | | | | | |
| | | 99.9 | 99.9 | 99.9 | | 99.9 | 99.9 | 99.9 | | 99.6 | 99.6 | 99.6 |
| | Impurities | | | | | | | | | | | |
| | | P1 | P2 | Avg | | P1 | P2 | Avg | | P1 | P2 | Avg |
| | RRT | (area %) | (area %) | (area %) | RRT | (area %) | (area %) | (area %) | RRT | (area %) | (area %) | (area %) |
| | — | — | — | — | — | — | — | — | — | — | — | — |
| | — | — | — | — | — | — | — | — | — | — | — | — |
| | — | — | — | — | — | — | — | — | 1.06 | NQ | NQ | NQ |
| | | | | | | | | | (Cmpd | | | |
| | | | | | | | | | 4) | | | |
| | 1.15 | 0.13 | 0.13 | 0.13 | 1.16 | 0.12 | 0.11 | 0.12 | 1.16 | 0.39 | 0.39 | 0.39 |
| | (Cmpd | | | | (Cmpd | | | | (Cmpd | | | |
| | 6) | | | | 6) | | | | 6) | | | |
| | — | — | — | — | — | — | — | — | — | — | — | — |
| | Total | 0.1 | 0.1 | 0.1 | Total | 0.1 | 0.1 | 0.1 | Total | 0.4 | 0.4 | 0.4 |
| Water | | <0.04 | <0.04 | <0.04 | | <0.04 | <0.04 | <0.04 | | <0.04 | 0.04 | 0.04 |
| Content (%) | | | | | | | | | | | | |

| | Test | | | | | | | |
|---|---|---|---|---|---|---|---|---|
| | 18 M | | | | 24 M | | | |
| | Prep | | | | | | | |
| | | P1 | P2 | Avg | | P1 | P2 | Avg |
| | Assay (%) | | | | | | | |
| | | 97.9 | 99.2 | 98.5 | | 100.2 | 99.3 | 99.7 |
| | Purity (%) | | | | | | | |
| | | 99.9 | 99.9 | 99.9 | | 99.9 | 99.9 | 99.9 |
| | Impurities | | | | | | | |
| | | P1 | P2 | Avg | | P1 | P2 | Avg |
| | RRT | (area %) | (area %) | (area %) | RRT | (area %) | (area %) | (area %) |
| | — | — | — | — | — | — | — | — |
| | — | — | — | — | — | — | — | — |
| | 1.06 | NQ | NQ | NA | 1.06 | NQ | NQ | NA |
| | 1.15 | 0.10 | 0.10 | 0.10 | 1.16 | 0.11 | 0.11 | 0.11 |
| | (Cmpd 6) | | | | (Cmpd 6) | | | |
| | — | — | — | — | — | — | — | — |
| | Total | 0.1 | 0.1 | 0.1 | Total | 0.1 | 0.1 | 0.1 |
| Water | | <0.04 | <0.04 | <0.04 | | <0.04 | <0.04 | <0.04 |
| Content (%) | | | | | | | | |

b) 25° C./60% RH:

| | Test | | | | | | | | | | | |
|---|---|---|---|---|---|---|---|---|---|---|---|---|
| | 0 M | | | | 2 M | | | | 3 M | | | |
| | Prep | | | | | | | | | | | |
| | | P1 | P2 | Avg | | P1 | P2 | Avg | | P1 | P2 | Avg |
| | Assay (%) | | | | | | | | | | | |
| | | 99.9 | 99.7 | 99.8 | | 98.9 | 99.3 | 99.1 | | 99.0 | 100.0 | 99.5 |
| | Purity (%) | | | | | | | | | | | |
| | | 99.8 | 99.8 | 99.8 | | 99.9 | 99.8 | 99.9 | | 99.7 | 99.7 | 99.7 |
| | Impurities | | | | | | | | | | | |
| | | P1 | P2 | Avg | | P1 | P2 | Avg | | P1 | P2 | Avg |
| | RRT | (area %) | (area %) | (area %) | RRT | (area %) | (area %) | (area %) | RRT | (area %) | (area %) | (area %) |
| | — | — | — | — | 0.71 | — | 0.08 | 0.08 | — | — | — | — |
| | — | — | — | — | 0.92 | — | NQ | NQ | — | — | — | — |
| | 0.99 | NQ | — | NQ | — | — | — | — | — | — | — | — |
| | 1.06 | — | NQ | NQ | — | — | — | — | 1.06 | 0.07 | 0.07 | 0.07 |
| | (Cmpd | | | | | | | | (Cmpd | | | |
| | 4) | | | | | | | | 4) | | | |
| | 1.17 | 0.13 | 0.12 | 0.13 | 1.16 | 0.05 | NQ | 0.05 | 1.16 | 0.22 | 0.22 | 0.22 |
| | (Cmpd | | | | (Cmpd | | | | (Cmpd | | | |
| | 6) | | | | 6) | | | | 6) | | | |
| | 1.32 | NQ | NQ | NQ | 1.29 | NQ | — | NQ | — | — | — | — |
| | Total | 0.1 | 0.1 | 0.1 | Total | 0.05 | 0.1 | 0.1 | Total | 0.3 | 0.3 | 0.3 |
| Water | | <0.04 | <0.04 | <0.04 | | <0.04 | <0.04 | <0.04 | | <0.04 | <0.04 | <0.04 |
| Content (%) | | | | | | | | | | | | |
| | Test | | | | | | | | | | | |
| | 6 M | | | | 9 M | | | | 12 M | | | |
| | Prep | | | | | | | | | | | |
| | | P1 | P2 | Avg | | P1 | P2 | Avg | | P1 | P2 | Avg |
| | Assay (%) | | | | | | | | | | | |
| | | 97.0 | 99.5 | 98.2 | | 99.7 | 97.0 | 98.4 | | 98.3 | 98.6 | 98.5 |
| | Purity (%) | | | | | | | | | | | |
| | | 99.9 | 99.9 | 99.9 | | 99.9 | 99.9 | 99.9 | | 99.6 | 99.6 | 99.6 |
| | Impurities | | | | | | | | | | | |
| | | P1 | P2 | Avg | | P1 | P2 | Avg | | P1 | P2 | Avg |
| | RRT | (area %) | (area %) | (area %) | RRT | (area %) | (area %) | (area %) | RRT | (area %) | (area %) | (area %) |
| | — | — | — | — | — | — | — | — | — | — | — | — |
| | — | — | — | — | — | — | — | — | — | — | — | — |
| | — | — | — | — | — | — | — | — | 1.06 | NQ | NQ | NQ |
| | | | | | | | | | (Cmpd | | | |
| | | | | | | | | | 4) | | | |
| | 1.15 | 0.11 | 0.12 | 0.11 | 1.16 | 0.11 | 0.10 | 0.10 | 1.16 | 0.39 | 0.39 | 0.39 |
| | (Cmpd | | | | (Cmpd | | | | (Cmpd | | | |
| | 6) | | | | 6) | | | | 6) | | | |
| | — | — | — | — | — | — | — | — | — | — | — | — |
| | — | — | — | — | — | — | — | — | 1.52 | NQ | — | NQ |
| | Total | 0.1 | 0.1 | 0.1 | Total | 0.1 | 0.1 | 0.1 | Total | 0.4 | 0.4 | 0.4 |
| Water | | <0.04 | <0.04 | <0.04 | | <0.04 | <0.04 | <0.04 | | <0.04 | 0.04 | 0.04 |
| Content (%) | | | | | | | | | | | | |

| | Test | | | | | | | |
|---|---|---|---|---|---|---|---|---|
| | 18 M | | | | 24 M | | | |
| | Prep | | | | | | | |
| | | P1 | P2 | Avg | | P1 | P2 | Avg |
| | Assay (%) | | | | | | | |
| | | 98.3 | 98.2 | 98.2 | | 99.1 | 99.6 | 99.3 |
| | Purity (%) | | | | | | | |
| | | 99.9 | 99.9 | 99.9 | | 99.9 | 99.9 | 99.9 |
| | Impurities | | | | | | | |
| | | P1 | P2 | Avg | | P1 | P2 | Avg |
| | RRT | (area %) | (area %) | (area %) | RRT | (area %) | (area %) | (area %) |
| | — | — | — | — | — | — | — | — |
| | — | — | — | — | — | — | — | — |
| | 1.06 | NQ | NQ | NA | 1.06 | NQ | NQ | NA |
| | 1.15 | 0.10 | 0.10 | 0.10 | 1.16 | 0.12 | 0.12 | 0.12 |
| | (Cmpd | | | | (Cmpd 6) | | | |
| | 6) | | | | | | | |
| | — | — | — | — | — | — | — | — |
| Water | Total | 0.1 | 0.1 | 0.1 | Total | 0.1 | 0.1 | 0.1 |
| Content (%) | | <0.04 | <0.04 | <0.04 | | <0.04 | <0.04 | <0.04 |

c) 40° C./75% RH:

| | Test | | | | | | | | | | | |
|---|---|---|---|---|---|---|---|---|---|---|---|---|
| | 0 M | | | | 2 M | | | | 3 M | | | |
| | Prep | | | | | | | | | | | |
| | | P1 | P2 | Avg | | P1 | P2 | Avg | | P1 | P2 | Avg |
| | Assay (%) | | | | | | | | | | | |
| | | 99.9 | 99.7 | 99.8 | | 100.0 | 99.8 | 99.9 | | 100.8 | 97.2 | 99.0 |
| | Purity (%) | | | | | | | | | | | |
| | | 99.8 | 99.8 | 99.8 | | 99.9 | 99.9 | 99.9 | | 99.7 | 99.7 | 99.7 |
| | Impurities | | | | | | | | | | | |
| | | P1 | P2 | Avg | | P1 | P2 | Avg | | P1 | P2 | Avg |
| | RRT | (area %) | (area %) | (area %) | RRT | (area %) | (area %) | (area %) | RRT | (area %) | (area %) | (area %) |
| | — | — | — | — | — | — | — | — | 0.36 | NQ | — | NQ |
| | 0.99 | NQ | — | NQ | 0.99 | NQ | NQ | NQ | — | — | — | — |
| | 1.06 | — | NQ | NQ | — | — | — | — | 1.06 | 0.07 | 0.07 | 0.07 |
| | (Cmpd | | | | | | | | (Cmpd | | | |
| | 4) | | | | | | | | 4) | | | |
| | 1.17 | 0.13 | 0.12 | 0.13 | 1.16 | 0.08 | 0.06 | 0.07 | 1.16 | 0.21 | 0.21 | 0.21 |
| | (Cmpd | | | | (Cmpd | | | | (Cmpd | | | |
| | 6) | | | | 6) | | | | 6) | | | |
| | 1.32 | NQ | NQ | NQ | 1.29 | — | NQ | NQ | — | — | — | — |
| | — | — | — | — | 1.38 | NQ | NQ | NQ | — | — | — | — |
| | Total | 0.1 | 0.1 | 0.1 | Total | 0.1 | 0.1 | 0.1 | Total | 0.3 | 0.3 | 0.3 |
| Water | | <0.04 | <0.04 | <0.04 | | <0.04 | <0.04 | <0.04 | | <0.04 | <0.04 | <0.04 |
| Content (%) | | | | | | | | | | | | |

| | Test | | | | | | | | | | | |
|---|---|---|---|---|---|---|---|---|---|---|---|---|
| | 6 M | | | | 8 M | | | | | | | |
| | Prep | | | | | | | | | | | |
| | | P1 | P2 | Avg | | P1 | P2 | P3 | P4 | P5 | P6 | Avg |
| | Assay (%) | | | | | | | | | | | |
| | | 98.5 | 95.9 | n/a | | 100.0 | 99.8 | 100.3 | 100.6 | 99.9 | 100.8 | 100.3 |
| | Purity (%) | | | | | | | | | | | |
| | | 99.9 | 99.9 | 99.9 | | 99.9 | 99.9 | 99.9 | 99.9 | 99.9 | 99.9 | 99.9 |
| | Impurities | | | | | | | | | | | |
| | | P1 | P2 | Avg | | P1 | P2 | Avg | | P1 | P2 | Avg |
| | RRT | (area %) | (area %) | (area %) | RRT | (area %) | (area %) | (area %) | RRT | (area %) | (area %) | (area %) |
| | — | — | — | — | — | — | — | — | — | — | — | — |
| | — | — | — | — | — | — | — | — | — | — | — | — |
| | 1.15 | 0.13 | 0.12 | 0.13 | 1.16 | 0.09 | 0.09 | 0.07 | 0.09 | 0.08 | 0.08 | 0.08 |
| | (Cmpd | | | | (Cmpd | | | | | | | |
| | 6) | | | | 6) | | | | | | | |
| | — | — | — | — | — | — | — | — | — | — | — | — |
| | Total | 0.1 | 0.1 | 0.1 | Total | 0.1 | 0.1 | 0.1 | 0.1 | 0.1 | 0.1 | 0.1 |
| Water | | <0.04 | <0.04 | <0.04 | | <0.04 | <0.04 | <0.04 | <0.04 | <0.04 | 0.04 | 0.04 |
| Content (%) | | | | | | | | | | | | |

### For Batch B:

a) Storage condition: 5° C.:

| | Test | | | | | | | | | | |
|---|---|---|---|---|---|---|---|---|---|---|---|
| | | | | | | | | 4.5 M (25° C./60% RH) + 1.5 M | | | |
| | 0 M | | | | | | | (5° C.) | | | |
| | Prep | | | | | | | | | | |
| | | P1 | P2 | P3 | P4 | P5 | Avg | | P1 | P2 | Avg |
| | Assay (%) | | | | | | | | | | |
| | | 99.0 | 100.5 | 99.3 | 99.5 | 98.8 | 99.4 | | 98.3 | 98.9 | 98.6 |
| | Purity (%) | | | | | | | | | | |
| | | 99.8 | 99.8 | 99.8 | 99.8 | 99.8 | 99.8 | | 99.6 | 99.5 | 99.6 |
| | Impurities | | | | | | | | | | |
| | | P1 | P2 | P3 | P4 | P5 | Avg | | P1 | P2 | Avg |
| | RRT | (area %) | (area %) | (area %) | (area %) | (area %) | (area %) | RRT | (area %) | (area %) | (area %) |
| | — | — | — | — | — | — | — | — | — | — | — |
| | 0.61 | NQ | NQ | 0.05 | 0.05 | 0.06 | 0.05 | — | — | — | — |
| | — | — | — | — | — | — | — | 0.80 | — | NQ | NQ |
| | 0.86 | NQ | NQ | NQ | NQ | NQ | NQ | 0.84 | NQ | NQ | NQ |
| | 0.88 | NQ | NP | NQ | NQ | NQ | NQ | 0.87 | NQ | NQ | NQ |
| | 0.99 | NQ | NQ | NQ | NQ | NQ | NQ | 0.99 | 0.05 | NQ | 0.05 |
| | — | — | — | — | — | — | — | 1.02 | NQ | NQ | NQ |
| | — | — | — | — | — | — | — | 1.05 | — | NQ | NQ |
| | — | — | — | — | — | — | — | 1.06 | NQ | NQ | NQ |
| | | | | | | | | (Cmpd | | | |
| | | | | | | | | 4) | | | |
| | — | — | — | — | — | — | — | — | — | — | — |
| | 1.16 | 0.08 | 0.07 | 0.08 | 0.07 | 0.08 | 0.07 | 1.17 | 0.27 | 0.25 | 0.26 |
| | (Cmp | | | | | | | (Cmp | | | |
| | 6) | | | | | | | 6) | | | |
| | — | — | — | — | — | — | — | 1.39 | NQ | NQ | NQ |
| | Total | 0.1 | 0.1 | 0.1 | 0.1 | 0.1 | 0.1 | Total | 0.3 | 0.2 | 0.3 |
| Water | | 0.1 | 0.1 | | | | 0.1 | | <0.04 | <0.04 | <0.04 |
| Content (%) | | | | | | | | | | | |

Test
9 M
Prep

| | | |
|---|---|---|
| P1 | P2 | Avg |

Assay (%)

| | | |
|---|---|---|
| 98.7 | 99.4 | 99.1 |

Purity (%)

| | | |
|---|---|---|
| 99.6 | 99.6 | 99.6 |

Impurities

| | | P1 | P2 | Avg |
|---|---|---|---|---|
| | RRT | (area %) | (area %) | (area %) |
| | — | — | — | — |
| | — | — | — | — |
| | — | — | — | — |
| | 0.84 | — | NQ | NQ |
| | 0.87 | NQ | NQ | NQ |
| | 0.99 | 0.11 | 0.11 | 0.11 |
| | 1.02 | NQ | NQ | NQ |
| | — | — | — | — |
| | — | — | — | — |
| | — | — | — | — |
| | 1.16 | 0.18 | 0.17 | 0.18 |
| | (Cmp | | | |
| | 6) | | | |
| | 1.39 | NQ | NQ | NQ |
| | 1.47 | — | NQ | NQ |
| | Total | 0.3 | 0.3 | 0.3 |
| Water | | 0.1 | 0.05 | 0.05 |
| Content (%) | | | | |

b) Storage condition: 25° C./60% RH:

| | Test | | | | | | | | | | |
|---|---|---|---|---|---|---|---|---|---|---|---|
| | 0 M | | | | | | | 1 M | | | |
| | Prep | | | | | | | | | | |
| | | P1 | P2 | P3 | P4 | P5 | Avg | | P1 | P2 | Avg |
| | Assay (%) | | | | | | | | | | |
| | | 99.0 | 100.5 | 99.3 | 99.5 | 98.8 | 99.4 | | 99.0 | 99.2 | 99.1 |
| | Purity (%) | | | | | | | | | | |
| | | 99.8 | 99.8 | 99.8 | 99.8 | 99.8 | 99.8 | | 99.4 | 99.4 | 99.4 |
| | Impurities | | | | | | | | | | |
| | | P1 | P2 | P3 | P4 | P5 | Avg | | P1 | P2 | Avg |
| | RRT | (area %) | (area %) | (area %) | (area %) | (area %) | (area %) | RRT | (area %) | (area %) | (area %) |
| | — | — | — | — | — | — | — | — | — | — | — |
| | 0.61 | NQ | NQ | 0.05 | 0.05 | 0.06 | 0.05 | — | — | — | — |
| | — | — | — | — | — | — | — | 0.80 | NQ | NQ | NQ |
| | 0.86 | NQ | NQ | NQ | NQ | NQ | NQ | 0.84 | NQ | NQ | NQ |
| | 0.88 | NQ | NP | NQ | NQ | NQ | NQ | 0.87 | NQ | NQ | NQ |
| | 0.99 | NQ | NQ | NQ | NQ | NQ | NQ | 0.99 | 0.10 | 0.10 | 0.10 |
| | — | — | — | — | — | — | — | 1.02 | NQ | NQ | NQ |
| | — | — | — | — | — | — | — | 1.05 | — | NQ | NQ |
| | — | — | — | — | — | — | — | 1.06 | NQ | — | NQ |
| | | | | | | | | (Cmpd | | | |
| | | | | | | | | 4) | | | |
| | 1.16 | 0.08 | 0.07 | 0.08 | 0.07 | 0.08 | 0.07 | 1.17 | 0.39 | 0.39 | 0.39 |
| | (Cmp | | | | | | | (Cmp | | | |
| | 6) | | | | | | | 6) | | | |
| | Total | 0.1 | 0.1 | 0.1 | 0.1 | 0.1 | 0.1 | Total | 0.5 | 0.5 | 0.5 |
| Water | | 0.1 | 0.1 | | | | 0.1 | | 0.1 | 0.1 | 0.1 |
| Content (%) | | | | | | | | | | | |

| | Test | | | | | | | |
|---|---|---|---|---|---|---|---|---|
| | 3 M | | | | 6 M | | | |
| | Prep | | | | | | | |
| | | P1 | P2 | Avg | | P1 | P2 | Avg |
| | Assay (%) | | | | | | | |
| | | 96.8 | 98.8 | 97.8 | | 99.0 | 98.5 | 98.7 |
| | Purity (%) | | | | | | | |
| | | 99.4 | 99.5 | 99.4 | | 99.6 | 99.5 | 99.5 |
| | Impurities | | | | | | | |
| | | P1 | P2 | Avg | | P1 | P2 | Avg |
| | RRT | (area %) | (area %) | (area %) | RRT | (area %) | (area %) | (area %) |
| | 0.48 | NQ | — | NQ | — | — | — | — |
| | — | — | — | — | — | — | — | — |
| | 0.80 | NQ | NQ | NQ | 0.80 | NQ | NQ | NQ |
| | 0.84 | NQ | NQ | NQ | 0.84 | NQ | NQ | NQ |
| | 0.87 | NQ | NQ | NQ | 0.87 | NQ | NQ | NQ |
| | 0.99 | 0.09 | 0.09 | 0.09 | 0.99 | NQ | 0.05 | 0.05 |
| | 1.02 | NQ | NQ | NQ | 1.02 | 0.05 | NQ | 0.05 |
| | 1.05 | — | — | — | 1.05 | NQ | NQ | NQ |
| | 1.06 | — | NQ | NQ | 1.06 | NQ | NQ | NQ |
| | (Cmpd 4) | | | | (Cmpd 4) | | | |
| | 1.17 | 0.33 | 0.33 | 0.33 | 1.17 | 0.23 | 0.25 | 0.24 |
| | (Cmp 6) | | | | (Cmp 6) | | | |
| | — | — | — | — | 1.39 | NQ | NQ | NQ |
| | Total | 0.4 | 0.4 | 0.4 | Total | 0.3 | 0.3 | 0.3 |
| Water | | 0.04 | 0.04 | 0.04 | | <0.04 | <0.04 | <0.04 |
| Content (%) | | | | | | | | |

Test
9 M
Prep

| | | |
|---|---|---|
| P1 | P2 | Avg |

Assay (%)

| | | |
|---|---|---|
| 99.7 | 98.2 | 99.0 |

Purity (%)

| | | |
|---|---|---|
| 99.6 | 99.5 | 99.5 |

Impurities

| | | P1 | P2 | Avg |
|---|---|---|---|---|
| | RRT | (area %) | (area %) | (area %) |
| | — | — | — | — |
| | — | — | — | — |
| | — | — | — | — |
| | 0.84 | NQ | NQ | NQ |
| | 0.87 | NQ | NQ | NQ |
| | 0.99 | 0.12 | 0.13 | 0.12 |
| | 1.02 | 0.06 | 0.06 | 0.06 |
| | — | — | — | — |
| | — | — | — | — |
| | — | — | — | — |
| | 1.16 | 0.18 | 0.18 | 0.18 |
| | (Cmp | | | |
| | 6) | | | |
| | 1.39 | NQ | NQ | NQ |
| | Total | 0.4 | 0.4 | 0.4 |
| Water | | 0.1 | 0.1 | 0.1 |
| Content (%) | | | | |

Storage condition: 40° C./75% RH:

| | Test | | | | | | | | | | |
|---|---|---|---|---|---|---|---|---|---|---|---|
| | 0 M | | | | | | | 1 M | | | |
| | Prep | | | | | | | | | | |
| | | P1 | P2 | P3 | P4 | P5 | Avg | | P1 | P2 | Avg |
| | Assay (%) | | | | | | | | | | |
| | | 99.0 | 100.5 | 99.3 | 99.5 | 98.8 | 99.4 | | 99.1 | 99.4 | 99.2 |
| | Purity (%) | | | | | | | | | | |
| | | 99.8 | 99.8 | 99.8 | 99.8 | 99.8 | 99.8 | | 99.3 | 99.3 | 99.3 |
| | Impurities | | | | | | | | | | |
| | | P1 | P2 | P3 | P4 | P5 | Avg | | P1 | P2 | Avg |
| | RRT | (area %) | (area %) | (area %) | (area %) | (area %) | (area %) | RRT | (area %) | (area %) | (area %) |
| | — | — | — | — | — | — | — | — | — | — | — |
| | 0.61 | NQ | NQ | 0.05 | 0.05 | 0.06 | 0.05 | — | — | — | — |
| | — | — | — | — | — | — | — | 0.80 | NQ | NQ | NQ |
| | 0.86 | NQ | NQ | NQ | NQ | NQ | NQ | 0.84 | NQ | NQ | NQ |
| | 0.88 | NQ | NP | NQ | NQ | NQ | NQ | 0.87 | NQ | NQ | NQ |
| | 0.99 | NQ | NQ | NQ | NQ | NQ | NQ | 0.99 | 0.14 | 0.14 | 0.14 |
| | — | — | — | — | — | — | — | 1.02 | 0.06 | 0.06 | 0.06 |
| | — | — | — | — | — | — | — | 1.05 | NQ | NQ | NQ |
| | — | — | — | — | — | — | — | 1.06 | NQ | NQ | NQ |
| | | | | | | | | (Cmpd | | | |
| | | | | | | | | 4) | | | |
| | 1.16 | 0.08 | 0.07 | 0.08 | 0.07 | 0.08 | 0.07 | 1.17 | 0.38 | 0.38 | 0.38 |
| | (Cmp | | | | | | | (Cmp | | | |
| | 6) | | | | | | | 6) | | | |
| | — | — | — | — | — | — | — | 1.39 | 0.05 | 0.05 | 0.05 |
| | Total | 0.1 | 0.1 | 0.1 | 0.1 | 0.1 | 0.1 | Total | 0.6 | 0.6 | 0.6 |
| Water | | 0.1 | 0.1 | | | | 0.1 | | 0.1 | 0.1 | 0.1 |
| Content (%) | | | | | | | | | | | |

| | Test | | | | | | | |
|---|---|---|---|---|---|---|---|---|
| | 3 M | | | | 6 M | | | |
| | Prep | | | | | | | |
| | | P1 | P2 | Avg | | P1 | P2 | Avg |
| | Assay (%) | | | | | | | |
| | | 98.5 | 99.0 | 98.7 | | 98.6 | 98.2 | 98.4 |
| | Purity (%) | | | | | | | |
| | | 98.9 | 98.9 | 98.9 | | 98.8 | 98.9 | 98.9 |
| | Impurities | | | | | | | |
| | | P1 | P2 | Avg | | P1 | P2 | Avg |
| | RRT | (area %) | (area %) | (area %) | RRT | (area %) | (area %) | (area %) |
| | 0.38 | NQ | NQ | NQ | 0.39 | NQ | NQ | NQ |
| | — | — | — | — | — | — | — | — |
| | 0.80 | NQ | NQ | NQ | 0.80 | NQ | NQ | NQ |
| | 0.84 | NQ | NQ | NQ | 0.84 | NQ | NQ | NQ |
| | 0.87 | NQ | NQ | NQ | 0.87 | NQ | NQ | NQ |
| | 0.99 | 0.24 | 0.24 | 0.24 | 0.99 | NQ | NQ | NQ |
| | 1.02 | 0.19 | 0.19 | 0.19 | 1.02 | 0.34 | 0.34 | 0.34 |
| | — | — | — | — | — | — | — | — |
| | 1.06 | NQ | — | NQ | 1.06 | NQ | NQ | NQ |
| | (Cmpd 4) | | | | (Cmpd 4) | | | |
| | 1.17 | 0.39 | 0.35 | 0.37 | 1.17 | 0.31 | 0.29 | 0.30 |
| | (Cmp 6) | | | | (Cmp 6) | | | |
| | | | | | 1.27 | NQ | — | NQ |
| | 1.39 | 0.16 | 0.16 | 0.16 | 1.39 | 0.34 | 0.34 | 0.34 |
| | Total | 1.0 | 0.9 | 1.0 | Total | 1.0 | 1.0 | 1.0 |
| Water | | <0.04 | 0.04 | 0.04 | | <0.04 | <0.04 | <0.04 |
| Content (%) | | | | | | | | |

Based on the stability tests for Batch A and Batch B above, Batch A has shown excellent stability over 24 months.

### 3-Month Stability Data for Batches B, C, D, and E

Batches B, C, and D were prepared according to Method 5, Method 4, and Method 3, respectively, as described in Example 1. Batch E was prepared analogously using according to Method 1 as described in Example 1.

Samples of TTI-101 (Batches B, C, D, and E) were stored at 40° C./75% RH for 3 months. The batches were individually stored in an low density polyethylene (LDPE) bag enclosed with a Nylon cable tie and then transferred to another LDPE bag and enclosed with a Nylon cable tie. The enclosure was put into a high density polyethylene (HDPE) bottle and closed with a polyethylene-lined polypropylene cap. For Batch B only, another sample was enclosed in a screw-top amber glass bottle and closed with a cap. The samples were stored under the above-identified conditions.

T = 0 month (0 M)

| | | | | |
|---|---|---|---|---|
| | | Batch B (LDPE | Batch B (glass | |
| Batch C | Batch D | storage) | storage) | Batch E |

Purity %

| | | | | |
|---|---|---|---|---|
| 99.6 | 99.9 | 99.6 | 99.6 | 99.9 |

Impurities

| RRT | Area % | RRT | Area % | RRT | Area % | RRT | Area % | RRT | Area % |
|---|---|---|---|---|---|---|---|---|---|
| — | — | — | — | 0.39 | 0.06 | 0.39 | 0.06 | — | — |
| — | — | — | — | 0.49 | NQ | 0.49 | NQ | — | — |
| — | — | — | — | 0.54 | NQ | 0.54 | NQ | — | — |
| 0.84 | NQ | — | — | — | — | — | — | — | — |
| 0.87 | NQ | — | — | — | — | — | — | — | — |
| 0.99 | 0.06 | — | — | 0.99 | NQ | 0.99 | NQ | — | — |
| 1.06 | NQ | — | — | — | — | — | — | — | — |
| (Cmp | | | | | | | | | |
| 4) | | | | | | | | | |
| 1.16 | 0.31 | 1.16 | 0.10 | 1.16 | 0.12 | 1.16 | 0.12 | 1.16 | 0.10 |
| (Cmp | | (Cmp | | (Cmp | | (Cmp | | (Cmp | |
| 6) | | 6) | | 6) | | 6) | | 6) | |
| Total | 0.4 | Total | 0.1 | Total | 0.2 | Total | 0.2 | Total | 0.1 |

T = 1 month (1 M)

| | | | | |
|---|---|---|---|---|
| | | Batch B (LDPE | Batch B (glass | |
| Batch C | Batch D | storage) | storage) | Batch E |

Purity %

| | | | | |
|---|---|---|---|---|
| 99.8 | 99.9 | 99.5 | 99.5 | 99.9 |

Impurities

| RRT | Area % | RRT | Area % | RRT | Area % | RRT | Area % | RRT | Area % |
|---|---|---|---|---|---|---|---|---|---|
| — | — | — | — | — | — | — | — | — | — |
| — | — | — | — | — | — | — | — | — | — |
| — | — | — | — | — | — | — | — | — | — |
| — | — | — | — | 0.80 | NQ | 0.80 | NQ | — | — |
| — | — | — | — | 0.84 | NQ | 0.84 | NQ | — | — |
| — | — | — | — | 0.87 | NQ | 0.87 | NQ | — | — |
| 0.99 | NQ | — | — | 0.99 | NQ | 0.99 | 0.05 | 0.99 | NQ |
| — | — | — | — | 1.02 | 0.05 | 1.02 | 0.06 | — | — |
| — | — | — | — | 1.05 | NQ | — | — | — | — |
| — | — | — | — | 1.06 | NQ | 1.06 | NQ | — | — |
| | | | | (Cmp | | (Cmp | | | |
| | | | | 4) | | 4) | | | |
| 1.17 | 0.12 | 1.17 | 0.09 | 1.17 | 0.24 | 1.17 | 0.23 | 1.17 | 0.09 |
| (Cmp | | (Cmp | | (Cmp | | (Cmp | | (Cmp | |
| 6) | | 6) | | 6) | | 6) | | 6) | |
| — | — | — | — | 1.39 | NQ | 1.39 | NQ | — | — |
| Total | 0.1 | Total | 0.1 | Total | 0.3 | Total | 0.3 | Total | 0.1 |

T = 2 months (2 M)

| | | Batch B (LDPE | Batch B (glass | |
|---|---|---|---|---|
| Batch C | Batch D | storage) | storage) | Batch E |

Purity %

| | | | | |
|---|---|---|---|---|
| 99.8 | 99.9 | 99.2 | 99.4 | 99.9 |

Impurities

| RRT | Area % | RRT | Area % | RRT | Area % | RRT | Area % | RRT | Area % |
|---|---|---|---|---|---|---|---|---|---|
| — | — | — | — | 0.39 | NQ | 0.39 | NQ | — | — |
| — | — | — | — | 0.51 | 0.06 | — | — | — | — |
| — | — | — | — | — | — | — | — | — | — |
| — | — | — | — | 0.80 | NQ | 0.80 | NQ | — | — |
| — | — | — | — | 0.84 | NQ | 0.84 | NQ | — | — |
| — | — | — | — | 0.87 | NQ | 0.87 | NQ | — | — |
| 0.99 | NQ | — | — | 0.99 | NQ | 0.99 | NQ | 0.99 | NQ |
| — | — | — | — | 1.02 | 0.11 | 1.02 | 0.10 | — | — |
| — | — | — | — | 1.05 | NQ | 1.05 | NQ | — | — |
| 1.06 | NQ | 1.06 | NQ | 1.06 | NQ | 1.06 | NQ | 1.06 | NQ |
| (Cmp | | (Cmp | | (Cmp | | (Cmp | | (Cmp | |
| 4) | | 4) | | 4) | | 4) | | 4) | |
| 1.17 | 0.13 | 1.17 | 0.09 | 1.17 | 0.32 | 1.17 | 0.23 | 1.17 | 0.09 |
| (Cmp | | (Cmp | | (Cmp | | (Cmp | | (Cmp | |
| 6) | | 6) | | 6) | | 6) | | 6) | |
| — | — | — | — | 1.39 | 0.1 | 1.39 | 0.09 | — | — |
| Total | 0.1 | Total | 0.1 | Total | 0.6 | Total | 0.4 | Total | 0.1 |

T = 3 months (3 M)

| | | Batch B (LDPE | Batch B (glass | |
|---|---|---|---|---|
| Batch C | Batch D | storage) | storage) | Batch E |

Purity %

| | | | | |
|---|---|---|---|---|
| 99.6 | 99.9 | 96.6 | 97.0 | 99.8 |

Impurities

| RRT | Area % | RRT | Area % | RRT | Area % | RRT | Area % | RRT | Area % |
|---|---|---|---|---|---|---|---|---|---|
| — | — | — | — | 0.40 | NQ | 0.40 | NQ | — | — |
| — | — | — | — | — | — | — | — | — | — |
| — | — | — | — | — | — | — | — | — | — |
| — | — | — | — | — | — | — | — | — | — |
| — | — | — | — | 0.85 | NQ | 0.85 | NQ | — | — |
| — | — | — | — | 0.88 | NQ | 0.88 | NQ | — | — |
| 0.95 | NQ | — | — | — | — | — | — | | |
| 0.98 | NQ | 0.98 | NQ | 0.98 | NQ | 0.98 | NQ | 0.98 | NQ |
| — | — | — | — | 1.02 | 0.13 | 1.02 | 0.13 | — | — |
| — | — | — | — | 1.05 | NQ | — | — | — | — |
| — | — | — | — | 1.06 | NQ | 1.06 | NQ | 1.06 | NQ |
| | | | | (Cmp | | (Cmp | | (Cmp | |
| | | | | 4) | | 4) | | 4) | |
| — | — | — | — | — | — | 1.10 | NQ | — | — |
| — | — | — | — | 1.13 | NQ | 1.13 | NQ | — | — |
| 1.16 | 0.31 | 1.16 | 0.11 | 1.16 | 2.9 | 1.16 | 2.6 | 1.16 | 0.16 |
| (Cmp | | (Cmp | | (Cmp | | (Cmp | | (Cmp | |
| 6) | | 6) | | 6) | | 6) | | 6) | |
| — | — | — | — | 1.38 | 0.16 | 1.39 | 0.14 | — | — |
| Total | 0.3 | Total | 0.1 | Total | 3.2 | Total | 2.9 | Total | 0.2 |

Storage condition: 40° C./75% relative humidity (RH)
Batch C:

| | | | |
|---|---|---|---|
| 0 M | 1 M | 2 M | 3 M |

Purity %

| | | | |
|---|---|---|---|
| 99.6 | 99.8 | 99.8 | 99.6 |

Impurities

| RRT | Area % | RRT | Area % | RRT | Area % | RRT | Area % |
|---|---|---|---|---|---|---|---|
| 0.84 | NQ | — | — | — | — | — | — |
| 0.87 | NQ | — | — | — | — | — | — |
| — | — | — | — | — | — | 0.95 | NQ |
| 0.99 | 0.06 | 0.99 | NQ | 0.99 | NQ | 0.98 | NQ |
| 1.06 | NQ | — | — | — | — | — | — |
| (Cmp 4) | | | | | | | |
| — | — | — | — | 1.06 | NQ | — | — |
| 1.16 | 0.31 | 1.17 | 0.12 | 1.16 | 0.13 | 1.16 | 0.31 |
| (Cmp 6) | | (Cmp 6) | | (Cmp 6) | | (Cmp 6) | |
| Total | 0.4 | Total | 0.1 | Total | 0.1 | Total | 0.3 |

Batch D:

| | | | |
|---|---|---|---|
| 0 M | 1 M | 2 M | 3 M |

Purity %

| | | | |
|---|---|---|---|
| 99.9 | 99.9 | 99.9 | 99.9 |

Impurities

| RRT | Area % | RRT | Area % | RRT | Area % | RRT | Area % |
|---|---|---|---|---|---|---|---|
| — | — | — | — | — | — | 0.98 | NQ |
| — | — | — | — | 1.06 | NQ | — | — |
| | | | | (Cmp 4) | | | |
| 1.16 | 0.10 | 1.17 | 0.09 | 1.17 | 0.09 | 1.16 | 0.11 |
| (Cmp 6) | | (Cmp 6) | | (Cmp 6) | | (Cmp 6) | |
| Total | 0.1 | Total | 0.1 | Total | 0.1 | Total | 0.1 |

Batch B (LDPE storage):

| | | | |
|---|---|---|---|
| 0 M | 1 M | 2 M | 3 M |

Purity %

| | | | |
|---|---|---|---|
| 99.8 | 99.5 | 99.2 | 96.6 |

Impurities

| RRT | Area % | RRT | Area % | RRT | Area % | RRT | Area % |
|---|---|---|---|---|---|---|---|
| 0.39 | 0.06 | — | — | 0.39 | NQ | 0.40 | NQ |
| 0.49 | NQ | — | — | 0.51 | 0.06 | — | — |
| 0.54 | NQ | — | — | — | — | — | — |
| — | — | 0.80 | NQ | 0.80 | NQ | — | — |
| — | — | 0.84 | NQ | 0.84 | NQ | 0.85 | NQ |
| — | — | 0.87 | NQ | 0.87 | NQ | 0.88 | NQ |
| 0.99 | NQ | 0.99 | NQ | 0.99 | NQ | 0.98 | NQ |
| — | — | 1.02 | 0.05 | 1.02 | 0.11 | 1.02 | 0.13 |
| — | — | 1.05 | NQ | 1.05 | NQ | 1.05 | NQ |
| — | — | 1.06 | NQ | 1.06 | NQ | 1.06 | NQ |
| | | (Cmp 4) | | (Cmp 4) | | (Cmp 4) | |
| — | — | — | — | — | — | 1.13 | NQ |
| 1.16 | 0.12 | 1.17 | 0.24 | 1.17 | 0.32 | 1.16 | 2.9 |
| (Cmp 6) | | (Cmp 6) | | (Cmp 6) | | (Cmp 6) | |
| — | — | 1.39 | NQ | 1.39 | 0.10 | 1.38 | 0.16 |
| Total | 0.2 | Total | 0.1 | Total | 0.6 | Total | 3.2 |

Batch B (glass storage):

| | | | |
|---|---|---|---|
| 0 M | 1 M | 2 M | 3 M |

Purity %

| | | | |
|---|---|---|---|
| 99.8 | 99.5 | 99.4 | 97.0 |

Impurities

| RRT | Area % | RRT | Area % | RRT | Area % | RRT | Area % |
|---|---|---|---|---|---|---|---|
| 0.39 | 0.06 | — | — | 0.39 | NQ | 0.40 | NQ |
| 0.49 | NQ | — | — | — | — | — | — |
| 0.54 | NQ | — | — | — | — | — | — |
| — | — | 0.80 | NQ | 0.80 | NQ | — | — |
| — | — | 0.84 | NQ | 0.84 | NQ | 0.85 | NQ |
| — | — | 0.87 | NQ | 0.87 | NQ | 0.88 | NQ |
| 0.99 | NQ | 0.99 | 0.05 | 0.99 | NQ | 0.98 | NQ |
| — | — | 1.02 | 0.06 | 1.02 | 0.10 | 1.02 | 0.13 |
| — | — | — | — | 1.05 | NQ | — | — |
| — | — | 1.06 | NQ | 1.06 | NQ | 1.06 | NQ |
| | | (Cmp 4) | | (Cmp 4) | | (Cmp 4) | |
| — | — | — | — | — | — | 1.10 | NQ |
| — | — | — | — | — | — | 1.13 | NQ |
| 1.16 | 0.12 | 1.17 | 0.23 | 1.17 | 0.323 | 1.16 | 2.6 |
| (Cmp 6) | | (Cmp 6) | | (Cmp 6) | | (Cmp 6) | |
| — | — | 1.39 | NQ | 1.39 | 0.09 | 1.38 | 0.14 |
| Total | 0.2 | Total | 0.3 | Total | 0.4 | Total | 2.9 |

Batch E:

| | | | |
|---|---|---|---|
| 0 M | 1 M | 2 M | 3 M |

Purity %

| | | | |
|---|---|---|---|
| 99.9 | 99.9 | 99.9 | 99.8 |

Impurities

| RRT | Area % | RRT | Area % | RRT | Area % | RRT | Area % |
|---|---|---|---|---|---|---|---|
| — | — | 0.99 | NQ | 0.99 | NQ | 0.98 | NQ |
| — | — | — | — | 1.06 | NQ | 1.06 | NQ |
| | | | | (Cmp 4) | | | |
| 1.16 | 0.10 | 1.17 | 0.09 | 1.17 | 0.09 | 1.16 | 0.16 |
| (Cmp 6) | | (Cmp 6) | | (Cmp 6) | | (Cmp 6) | |
| Total | 0.1 | Total | 0.1 | Total | 0.1 | Total | 0.2 |

1. A composition, comprising a compound of formula (I): 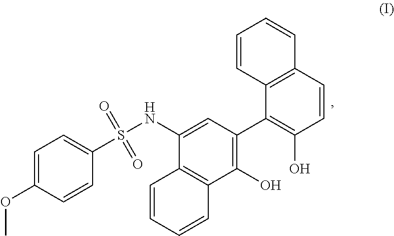 [SMILES: COc1ccc(S(=O)(=O)Nc2cc(-c3c(O)ccc4ccccc34)c(O)c3ccccc23)cc1]
wherein the composition is substantially free of an impurity selected from the group consisting of a compound of formula (II), a compound of formula (III), and a compound of formula (IV), or a combination thereof: 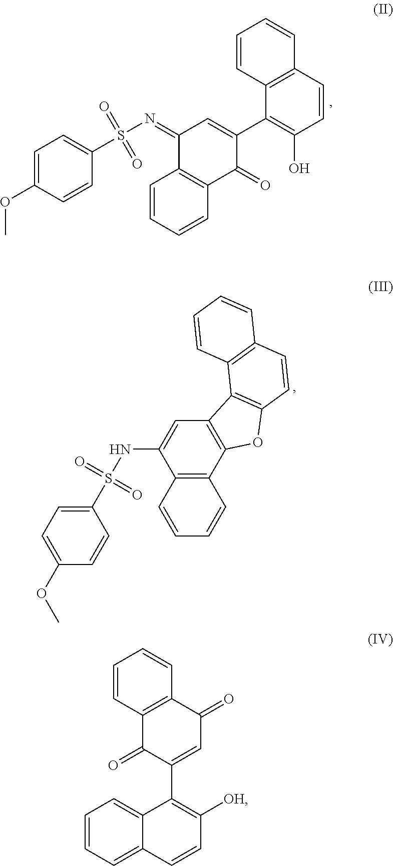 [SMILES: COc1ccc(S(=O)(=O)/N=C2/C=C(c3c(O)ccc4ccccc34)C(=O)c3ccccc32)cc1 ; COc1ccc(S(=O)(=O)Nc2cc3c(oc4ccc5ccccc5c43)c3ccccc23)cc1 ; O=C1C=C(c2c(O)ccc3ccccc23)C(=O)c2ccccc21]
wherein the impurity is present in the composition at less than about 1% area percentage by high-performance liquid chromatography (HPLC).

2. The composition of claim 1, wherein the impurity is present in the composition at less than about 0.5% area percentage by HPLC.

3. The composition of claim 1, wherein the impurity is present in the composition at less than about 0.1% area percentage by HPLC.

4. The composition of claim 1, wherein the impurity is present in the composition at less than about 0.05% area percentage by HPLC.

5. The composition of claim 1, wherein the amount of the impurity is undetectable by HPLC.

6. A pharmaceutical composition, comprising
a) a composition of claim 1, and
b) a pharmaceutically acceptable carrier.

7. A method of treating cancer in an individual in need thereof, the method comprising administering to the individual an effective amount of the composition of claim 1.

8. The method of claim 7, wherein the cancer is head and neck cancer, lung cancer, liver cancer, breast cancer, skin cancer, testicular cancer, colon cancer, rectal cancer, gastric cancer, bone cancer, spleen cancer, gall bladder cancer, stomach cancer, anal cancer, post-transplant lymphoproliferative disease, restenosis, ovarian cancer, colon cancer, multiple myeloma, prostate cancer, cervical cancer, brain cancer, pancreatic cancer, myelodysplastic syndrome, leukemia, lymphoma, neuroblastoma, kidney cancer, or metastatic melanoma.

9. The method of claim 7, wherein the cancer is head and neck cancer, lung cancer, liver cancer, breast cancer, ovarian cancer, colon cancer, multiple myeloma, leukemia, or pancreatic cancer.

10. A method of treating fibrosis in an individual in need thereof, the method comprising administering to the individual an effective amount of the composition of claim 1.

11. The method of claim 10, wherein the fibrosis is associated with pulmonary fibrosis, intestine fibrosis, pancreatic fibrosis, joint fibrosis, liver fibrosis, retroperitoneal fibrosis, myelofibrosis, renal fibrosis, bone marrow fibrosis, dermal fibrosis, non-alcoholic fatty liver disease, steatohepatitis, or systemic sclerosis.

12. The method of claim 10, wherein the fibrosis is associated with pulmonary fibrosis, non-alcoholic fatty liver disease, steatohepatitis, or systemic sclerosis.

13. A method of treating inflammatory disease or disorder in an individual in need thereof, the method comprising administering to the individual an effective amount of the composition of claim 1.

14. The method of claim 13, wherein the inflammatory disease or disorder is inflammatory bowel disease, ulcerative colitis, psoriasis, uveitis, scleritis, multiple sclerosis, pancreatitis, or asthma.

15. A method of treating neurodegenerative disease or disorder in an individual in need thereof, comprising administering to the subject an effective amount of the composition of claim 1.

16. The method of claim 15, wherein the neurodegenerative disease or disorder is chemotherapy-induced peripheral neuropathy, diabetic neuropathy, or familial amyloid polyneuropathy.

17. A method of treating non-alcoholic fatty liver disease or steatohepatitis in an individual in need thereof, comprising administering to the subject an effective amount of the composition of claim 1.

18. A method of treating cachexia in an individual in need thereof, comprising administering to the subject an effective amount of the composition of claim 1.

19. A method of treating anaphylaxis in an individual in need thereof, comprising administering to the subject an effective amount of the composition of claim 1.
